(19) United States
(12) Patent Application Publication
(10) Pub. No.: US 2026/0240798 A1
(43) Pub. Date: Aug. 20, 2026

(54) SOLID FORMS OF RXFP1 MODULATORS

(71) Applicants: ASTRAZENECA AB, Sodertalje (SE); MITSUBISHI TANABE PHARMA CORPORATION, Chuo-ku, Osaka-shi, Osaka (JP)

(72) Inventors: Inge Torbjörn ARVIDSSON, Sodertalje (SE); Marika Jenny Susanna LINDHAGEN, Sodertalje (SE); Okky Dwichandra PUTRA, Sodertalje (SE); Hosam Al-Deen ABU AWWAD, Cambridge (GB); Philip Anthony CORNER, Cambridge (GB); Dhaval Rasikbhai KALARIA, Cambridge (GB)

(21) Appl. No.: 18/872,328

(22) PCT Filed: Jun. 6, 2023
(86) PCT No.: PCT/EP2023/065044

§ 371 Date: Dec. 6, 2024

Related U.S. Application Data
(60) Provisional application No. 63/365,970, filed on Jun. 7, 2022.

(51) Int. Cl. A61K 31/192; A61K 9/16; C07C 237/24

(52) U.S. Cl. CPC A61K 31/192; A61K 9/1635; A61K 9/1647; A61K 9/1652; C07C 237/24

(57) ABSTRACT

The specification generally relates to solid forms, for example, crystalline and amorphous forms, of (1S,4s)-4-(2-fluoro-4-methoxy-5-(((1S,2R,3S,4R)-3-(((1-methylcyclobutyl)methyl)carbamoyl)bicyclo[2.2.1]heptan-2-yl)carbamoyl)phenoxy)-1-methylcyclohexane-1-carboxylic acid (Compound (I)). In particular, an amorphous form of Compound (I), solid dispersions and pharmaceutical compositions comprising such an amorphous form, and crystalline Form G of Compound (I).

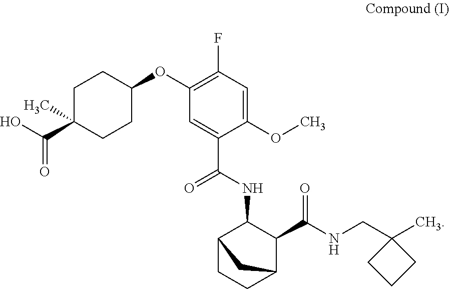 [SMILES: COc1cc(F)c(O[C@H]2CC[C@@](C)(C(=O)O)CC2)cc1C(=O)N[C@@H]1[C@H]2CC[C@H](C2)[C@@H]1C(=O)NCC1(C)CCC1]

### FIELD

Described in this specification are solid forms, for example, crystalline and amorphous forms, of the RXFP1 modulator (1S,4s)-4-(2-fluoro-4-methoxy-5-(((1S,2R,3S,4R)-3-(((1-methylcyclobutyl)methyl)carbamoyl)bicyclo[2.2.1]heptan-2-yl)carbamoyl)phenoxy)-1-methylcyclohexane-1-carboxylic acid (referred to as Compound (I) herein), pharmaceutical compositions thereof and processes for preparing such solid forms.

### BACKGROUND

Relaxin is a pleiotropic hormone known to mediate systemic haemodynamic and renal adaptive changes during pregnancy. Relaxin has also been shown to have anti-fibrotic properties and to have beneficial effects in heart failure e.g. with acute decompensated heart failure (ADHF). Heart failure is associated with significant morbidity and mortality. It is characterized by complex tissue remodelling involving increased cardiomyocyte death and interstitial fibrosis. Relaxin activates a number of signalling cascades which have been shown to be beneficial in the setting of ischemia-reperfusion and heart failure. These signalling pathways include activation of the phosphoinositide 3-kinase pathway and activation of the nitric oxide signalling pathway (Bathgate R A et al. (2013) Physiol. Rev. 93(1): 405-480; Mentz R J et al. (2013) Am. Heart J. 165(2): 193-199; Tietjens J et al. (2016) Heart 102:95-99; Wilson S S et al. (2015) Pharmacology 35:315-327).

In heart failure patients, a significant subset also suffer from pulmonary hypertension (HF+PH patients). It was estimated that approximately 50% of heart failure patients with preserved ejection fraction also suffer from pulmonary hypertension, increasing to 60% of heart failure patients with reduced ejection fraction (Guazzi, (2014) Circ Heart Fail., 7:367-377; Miller et al., (2013) JACC Heart Fail., 1(4):290-299). Patients suffering from heart failure with pulmonary hypertension have been shown to have reduced survival as compared with heart failure patients without pulmonary hypertension (Barnett and De Marco, (2012) Heart Fail. Clin. 8:447-459). In heart failure patients, a 3 mmHg increase or decrease in Estimated Pulmonary Artery Diastolic Pressure (ePAD), equivalent to approximately 4 mmHg increase or decrease in mean Pulmonary Arterial Pressure (mPAP), was associated with a 24% increase or a 19% decrease in cardiovascular mortality respectively (Zile M R, et al. (2017) Circ Heart Fail., 10:e003594). A 4 mmHg reduction in mPAP is also associated with dyspnea improvement in patients suffering from heart failure and pulmonary hypertension (Solomonica A, et al. (2013) Circ Heart Fail., 6:53-60).

Resistant hypertension (rHT) is defined as the blood pressure of a hypertensive patient that remains elevated above target goal despite the concurrent use of optimized doses of 3 antihypertensive agents of different classes, one of which is a diuretic. Current SoC for the initial treatment of hypertension is a calcium channel blocker (CCB), a blocker of the renin-angiotensin system (angiotensin-converting enzyme [ACE] inhibitor or angiotensin receptor blocker [ARB]), and a diuretic. For patients with rHT, there are multiple options for what to add next (such as a mineralocorticoid-receptor antagonist (MRA), beta-blocker, or alpha-blocker) and guidelines currently recommend a MRA as preferred option for treatment of rHT. rHT also includes patients whose blood pressure is adequately controlled when receiving 4 or more antihypertensive medications concurrently (Carey et al., Hypertension, 2018, 72, e53-e90). Patients with rHT typically have long histories of severe blood pressure elevation, predisposing them to higher cardiovascular risk than treated hypertensive patients with controlled blood pressure (Acelajado et al., Circulation Research, 2019, 124, 1061-1070). It has been suggested that relaxin may have therapeutic potential for hypertensive disease (Lekgabe et al., Hypertension, 2005, 46, 412-8).

Clinical trials have been conducted using unmodified recombinant human Relaxin-2, serelaxin. Continuous intravenous administration of serelaxin to hospitalized patients improved the markers of cardiac, renal and hepatic damage and congestion (Felker G M et al. (2014) J. Am. Coll. Cardiol. 64(15): 1591-1598; Metra M et al. (2013) J. Am. Coll. Cardiol. 61(2): 196-206; Teerlink J R et al. (2013) Lancet 381 (9860): 29-39). However, due to the rapid clearance of serelaxin from the patients' circulation, the therapeutic effects were limited and the positive effects rapidly disappeared once intravenous injection stopped. Additionally, approximately one third of the patients experienced a significant blood pressure drop (>40 mm Hg) after receiving serelaxin intravenously, with the consequence that the dose had to be reduced by half or even more.

The cognate receptor for human relaxin is RXFP1 and is a well-validated pharmacologically important GPCR family 1c member whose activation by the hormone relaxin is associated with hemodynamic, anti-fibrotic and anti-inflammatory properties (Halls M L et al., (2015), Pharmacol Rev. 67(2): 389-440).

Small-molecule modulators of RXFP1 have been sought as relaxin mimetics. For example, Marugan, J. J., et al., WO2013/165606A1; Xiao J et al. (2013) Nat. Commun. 4:1953; and McBride A et al. (2017) Scientific Reports 7:10806 discuss small-molecule modulators of RXFP1.

Despite the foregoing, a need continues to exist for further compounds, such as Compound (I), that are modulators of RXFP1 which may make the compounds especially promising for development as therapeutic agents. Such compounds may also exhibit improved modulation of RXFP1 in comparison with other known RXFP1 modulators. Such compounds may also exhibit favourable pharmacokinetic profiles (for example, lower intrinsic clearance) and/or advantageous physical properties (for example, higher aqueous solubility) in comparison with other known RXFP1 modulators. Therefore, such compounds may be especially useful in the treatment of disease states in which modulation of RXFP1 is beneficial. There is also a need for stable solid forms of such compounds with advantageous physical properties such as aqueous solubility at low pH which may be useful for oral administration of such compounds.

### SUMMARY

This specification relates to solid forms of Compound (I) which has the structure below:

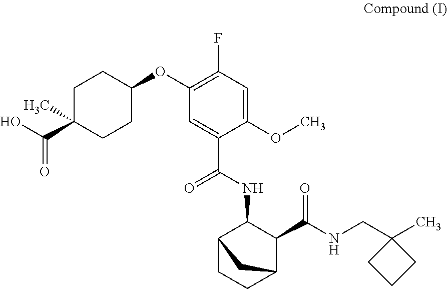 [SMILES: COc1cc(F)c(O[C@H]2CC[C@@](C)(C(=O)O)CC2)cc1C(=O)N[C@@H]1[C@H]2CC[C@H](C2)[C@@H]1C(=O)NCC1(C)CCC1]

and has the chemical name: (1S,4s)-4-(2-fluoro-4-methoxy-5-(((1S,2R,3S,4R)-3-(((1-methylcyclobutyl)methyl)carbamoyl)bicyclo[2.2.1]heptan-2-yl)carbamoyl)phenoxy)-1-methylcyclohexane-1-carboxylic acid.

Briefly, this specification describes, in part, amorphous Compound (I).

This specification also describes, in part, a pharmaceutical composition comprising amorphous Compound (I) and a pharmaceutically acceptable excipient.

This specification also describes, in part, a solid dispersion comprising amorphous Compound (I).

This specification also describes, in part, a pharmaceutical composition comprising a solid dispersion comprising amorphous Compound (I).

This specification also describes, in part, amorphous Compound (I) or the solid dispersions or pharmaceutical compositions described herein for use in therapy.

This specification also describes, in part, amorphous Compound (I) or the solid dispersions or pharmaceutical compositions described herein for use in the treatment of a subject with a disease or condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension.

This specification also describes, in part, a crystalline form of Compound (I), wherein the crystalline form has a melting onset at about 217.5° C., and a method of preparing such a crystalline form comprising heating crystalline Form A of Compound (I) to a temperature above about 43.9° C.

This specification also describes, in part, a crystalline form of Compound (I), obtainable by heating crystalline Form A of Compound (I) to a temperature above about 43.9° C., and a method of preparing such a crystalline form comprising heating crystalline Form A of Compound (I) to a temperature above about 43.9° C.

This specification also describes, in part, a pharmaceutical composition comprising the crystalline form as disclosed herein.

This specification also describes, in part, a crystalline form as disclosed herein for use in the manufacture of a medicament for the treatment of a disease or condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension in a human patient.

Further aspects of the disclosure will be apparent to one skilled in the art from reading this specification.

### LIST OF FIGURES

FIG. 1 is an X-Ray Powder Diffraction Pattern of Form A of Compound (I).

FIG. 2 is an X-Ray Powder Diffraction Pattern of amorphous Compound (I).

FIG. 3 is an X-Ray Powder Diffraction Pattern of amorphous solid dispersions of Compound (I) (20%, 30% and 40% loading of Compound (I)) after formation, after storage for 4 weeks at 40° C./75% relative humidity, and after storage for 4 weeks at 50° C. at ambient relative humidity.

FIG. 4 shows the DSC output from analysis of Form A/Form G of Compound (I).

FIG. 5 shows the TGA analysis of Form A/Form G of Compound (I).

FIG. 6 shows the light scattering intensity as a function of total concentration for amorphous Compound (I) in pH 1.2 simulated gastric fluid.

### DESCRIPTION OF ILLUSTRATIVE EMBODIMENTS

Many embodiments are detailed throughout the specification and will be apparent to a reader skilled in the art. The specification is not to be interpreted as being limited to any particular embodiment(s) described herein.

Terms not specifically defined herein should be understood to have the meanings that would be given to them by one of skill in the art in light of the disclosure and the context.

“About” may generally mean an acceptable degree of error for the quantity measured given the nature or precision of the measurements. Exemplary degrees of error are within percent (%), typically, within 10%, and more typically, within 5% of a given value or range of values.

Embodiments described herein as “comprising” one or more features may also be considered as disclosure of the corresponding embodiments “consisting of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

The chemical names of compounds described in this specification were generated using ChemDraw® Professional version 19.0.0.22 from PerkinElmer®. The skilled person will understand that different chemical naming software may generate different chemical names for a particular compound. In case a compound described herein is depicted in form of a chemical name and as a formula, the formula shall prevail in case of any discrepancy.

Compounds and salts described in this specification may exist in solvated forms and unsolvated forms. For example, a solvated form may be a hydrated form, such as a hemi-hydrate, a mono-hydrate, a di-hydrate, a tri-hydrate or an alternative quantity thereof. All such solvated and unsolvated forms of compounds described herein are encompassed herein.

Atoms of the compounds and salts described in this specification may exist as their isotopes. All compounds described herein where an atom is replaced by one or more of its isotopes (for example a compound described herein where one or more carbon atom is an ¹¹C or ¹³C carbon isotope, or where one or more hydrogen atoms is a ²H or ³H isotope) are encompassed herein.

Compounds described herein may exist in one or more geometrical, optical, enantiomeric, and diastereomeric forms, including, but not limited to, cis- and trans-forms, E- and Z-forms, and R-, S- and meso-forms. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers can be prepared by the application or adaptation of known methods.

The compounds described herein may include one or more chiral centres. To the extent a structure or chemical name in this specification does not indicate chirality, the structure or name is intended to encompass any single stereoisomer corresponding to that structure or name, as well as any mixture of stereoisomers (e.g. a racemate). Where a structure in this specification includes bonds drawn as solid and hashed wedges (i.e. [symbol] and [symbol] ), it is intended that the solid and hashed wedges indicate the absolute configuration of a chiral centre.

It is well-known in the art how such optically-active forms can be separated. For example, a single stereoisomer can be obtained by isolating it from a mixtures of isomers (e.g. a racemate) using, for example, chiral chromatographic separation. In other embodiments, a single stereoisomer is obtained through direct synthesis from, for example, a chiral starting material.

According to one embodiment, the compounds described herein are provided as a single enantiomer being in enantiomer excess (% ee) of ≥95%, ≥98%, or ≥99%. Conveniently a single enantiomer is present in an enantiomer excess of ≥99%.

According to one embodiment, a compound described herein is provided as a single enantiomer being in enantiomer excess (% ee) in the range 95 to 100%.

Compounds described herein may exist in one or more tautomeric forms, including, but not limited to, keto-, and enol-forms. A reference to a particular compound includes all tautomeric forms, including mixtures thereof. Accordingly, a structure depicted herein as one tautomer is intended to also include other tautomers.

The pharmaceutical compositions described herein may include one or more pharmaceutically acceptable excipients. The excipient(s) selected for inclusion in a particular composition will depend on factors such as the mode of administration and the form of the composition provided. Suitable pharmaceutically acceptable excipients are well known to persons skilled in the art and are described, for example, in the Handbook of Pharmaceutical Excipients, Sixth edition, Pharmaceutical Press, edited by Rowe, Ray C; Sheskey, Paul J; Quinn, Marian. Pharmaceutically acceptable excipients may function as, for example, adjuvants, diluents, carriers, stabilisers, flavourings, colorants, fillers, binders, disintegrants, lubricants, glidants, thickening agents and coating agents. As persons skilled in the art will appreciate, certain pharmaceutically acceptable excipients may serve more than one function and may serve alternative functions depending on how much of the excipient is present in the composition and what other excipients are present in the composition.

The pharmaceutical compositions may be in a form suitable for oral use (for example as tablets, lozenges, hard or soft capsules, aqueous or oily suspensions, or dispersible powders or granules), for topical use (for example as creams, ointments, or aqueous or oily suspensions), for administration by inhalation (for example as a finely divided powder), for administration by insufflation (for example as a finely divided powder), or as a suppository for rectal dosing. The compositions may be obtained by conventional procedures well known in the art. Compositions intended for oral use may contain additional components, for example, one or more colouring, sweetening, flavouring and/or preservative agents.

### Amorphous Compound (I)

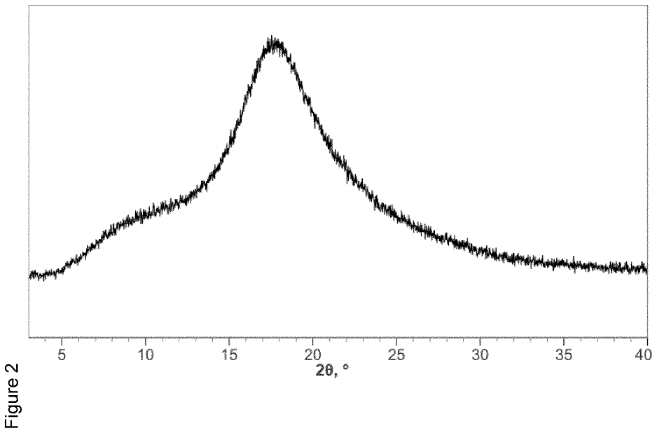

In an embodiment there is provided amorphous Compound (I). In one embodiment, the amorphous Compound (I) has an X-ray powder diffraction pattern (Cu K_{α} radiation) substantially the same as the X-ray powder diffraction pattern shown in FIG. 2. In one embodiment, the amorphous Compound (I) shows no reflections in the range of from 3 to 40° 2-Theta (Cu K_{α} radiation).

### Pharmaceutical Compositions Comprising Amorphous Compound (I)

In an embodiment there is provided a pharmaceutical composition comprising amorphous Compound (I) and a pharmaceutically acceptable excipient. In one embodiment, at least 80% by weight of Compound (I) in the pharmaceutical composition is amorphous. In one embodiment, at least 90% by weight of Compound (I) in the pharmaceutical composition is amorphous. In one embodiment, at least 95% by weight of Compound (I) in the pharmaceutical composition is amorphous. In one embodiment, at least 99% by weight of Compound (I) in the pharmaceutical composition is amorphous. In one embodiment, substantially all of the Compound (I) in the pharmaceutical composition is amorphous. In one embodiment, all of the Compound (I) in the pharmaceutical composition is amorphous.

In one embodiment, less than 20% by weight of Compound (I) in the pharmaceutical composition is crystalline. In one embodiment, less than 10% by weight of Compound (I) in the pharmaceutical composition is crystalline. In one embodiment, less than 5% by weight of Compound (I) in the pharmaceutical composition is crystalline. In one embodiment, less than 1% by weight of Compound (I) in the pharmaceutical composition is crystalline. In one embodiment, substantially none of the Compound (I) in the pharmaceutical composition is crystalline. In one embodiment, none of the Compound (I) in the pharmaceutical composition is crystalline.

### Solid Dispersions Comprising Amorphous Compound (I)

In another embodiment there is provided a solid dispersion comprising amorphous Compound (I). Solid dispersions typically include a compound dispersed in an appropriate carrier medium, such as one or more polymers. In one embodiment, the solid dispersion further comprises one or more polymers. In one embodiment, the one or more polymers are water-soluble. It would be understood that a water-soluble polymer is able to dissolve in an aqueous medium such as water or gastric fluid.

In one embodiment, at least 90% by weight of Compound (I) in the solid dispersion is amorphous. In one embodiment, at least 95% by weight of Compound (I) in the solid dispersion is amorphous. In one embodiment, at least 99% by weight of Compound (I) in the solid dispersion is amorphous. In one embodiment, substantially all of the Compound (I) in the solid dispersion is amorphous. In one embodiment, all of the Compound (I) in the solid dispersion is amorphous.

In one embodiment, less than 20% by weight of Compound (I) in the solid dispersion is crystalline. In one embodiment, less than 10% by weight of Compound (I) in the solid dispersion is crystalline. In one embodiment, less than 5% by weight of Compound (I) in the solid dispersion is crystalline. In one embodiment, less than 1% by weight of Compound (I) in the solid dispersion is crystalline. In one embodiment, substantially none of the Compound (I) in the solid dispersion is crystalline. In one embodiment, none of the Compound (I) in the solid dispersion is crystalline.

In one embodiment, Compound (I) is present in the solid dispersion in an amount of from 5% by weight to 95% by weight. In one embodiment, Compound (I) is present in the solid dispersion in an amount of from 5% by weight to 80% by weight. In one embodiment, Compound (I) is present in the solid dispersion in an amount of from 5% by weight to 60% by weight. In one embodiment, Compound (I) is present in the solid dispersion in an amount of from 10% by weight to 40% by weight. In one embodiment, Compound (I) is present in the solid dispersion in an amount of from 20% by weight to 40% by weight.

In one embodiment, the one or more polymers are selected from poly(1-vinyl-2-pyrrolidone), a copolymer of 1-vinyl-2-pyrrolidone and vinyl acetate, polyvinyl caprolactam-polyvinyl acetate-polyethylene glycol graft co-polymer, methacrylic acid-ethyl acrylate copolymer, polyacrylic acid, hypromellose acetate succinate, cellulose acetate phthalate, and polyvinyl acetate phthalate.

In one embodiment, the one or more polymers are selected from Kollidon® 30, Kollidon® VA 64, Soluplus®, Eudragit® L100-55S, Polyacrylic acid, AQOAT® HPMC AS-LF, AQOAT® HPMC AS-HF, Cellulose acetate phthalate and Polyvinyl Acetate Phthalate.

In one embodiment, the one or more polymers are a copolymer of 1-vinyl-2-pyrrolidone and vinyl acetate. In one embodiment, the one or more polymers are a copolymer of 1-vinyl-2-pyrrolidone and vinyl acetate in a ratio of 6:4 by weight. In one embodiment, the one or more polymers are Kollidon® VA 64.

In one embodiment, the one or more polymers are present in the solid dispersion in an amount of from 5% by weight to 95% by weight. In one embodiment, the one or more polymers are present in the solid dispersion in an amount of from 20% by weight to 90% by weight. In one embodiment, the one or more polymers are present in the solid dispersion in an amount of from 40% by weight to 90% by weight. In one embodiment, the one or more polymers are present in the solid dispersion in an amount of from 50% by weight to 90% by weight. In one embodiment, the one or more polymers are present in the solid dispersion in an amount of from 60% by weight to 80% by weight.

### Pharmaceutical Compositions Comprising Solid Dispersions Comprising Amorphous Compound (I)

In an embodiment there is provided a pharmaceutical composition comprising a solid dispersion comprising amorphous Compound (I), as described herein.

### Crystalline Form G of Compound (I)

Differential Scanning calorimetry (DSC) analysis of crystalline Form A of Compound (I) shows an endothermic transformation with an onset observed around 43.9° C. This endothermic peak is assigned to a solid-solid phase transition and the resulting phase at temperatures above the completion of the endothermic peak observed at around 43.9° C. is referred to as Form G. This, new, anhydrous phase Form G exists until the last endothermic peak in the DSC which corresponds to melting onset at 217.5° C.

Accordingly, in one embodiment is provided a crystalline form (Form G) of Compound (I), wherein the crystalline form has a melting onset at about 217.5° C. In one embodiment, the melting onset is 217.5±5° C. In one embodiment, the melting onset is 217.5±4° C. In one embodiment, the melting onset is 217.5±3° C. In one embodiment, the melting onset is 217.5±2° C. In one embodiment, the melting onset is 217.5±1° C.

In one embodiment, a crystalline form (Form G) of Compound (I) is obtainable by heating crystalline Form A of Compound (I) to a temperature above about 43.9° C. In one embodiment, the crystalline form is obtainable by heating crystalline Form A of Compound (I) to a temperature above about 45° C. In one embodiment, the crystalline form is obtainable by heating crystalline Form A of Compound (I) to a temperature above about 50° C. In one embodiment, the crystalline form is obtainable by heating crystalline Form A of Compound (I) to a temperature above about 55° C. In one embodiment, the crystalline form is obtainable by heating crystalline Form A of Compound (I) to a temperature above about 60° C. In one embodiment, the crystalline form is obtainable by heating crystalline Form A of Compound (I) to a temperature above about 65° C.

In one embodiment is provided a method of preparing a crystalline form (Form G) of Compound (I), comprising heating crystalline Form A of Compound (I) to a temperature above about 43.9° C. In one embodiment, crystalline Form A of Compound (I) is heated to a temperature above about 45° C. In one embodiment, crystalline Form A of Compound (I) is heated to a temperature above about 50° C. In one embodiment, crystalline Form A of Compound (I) is heated to a temperature above about 55° C. In one embodiment, crystalline Form A of Compound (I) is heated to a temperature above about 60° C. In one embodiment, crystalline Form A of Compound (I) is heated to a temperature above about 65° C.

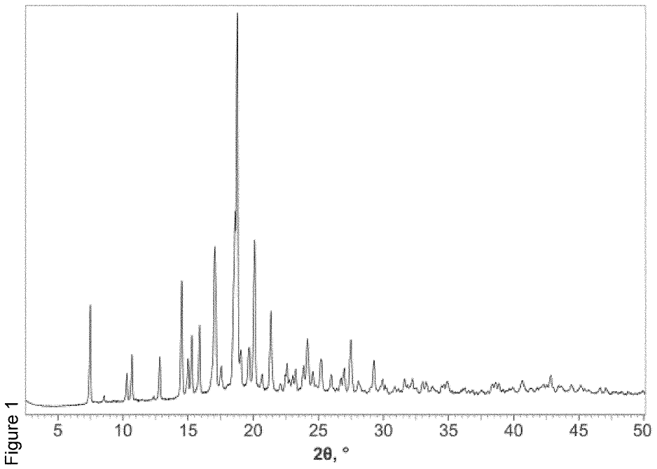

The ten most prominent X-Ray powder diffraction peaks for Form A of Compound (I) [Angle 2-theta (2θ), Intensity] are: 7.5 (s), 10.3 (m), 10.7 (s), 12.8 (s), 14.5 (vs), 15.3 (s), 15.8 (s), 17.5 (m), 19.6 (s) and 21.3 (s). In one embodiment, Form A of Compound (I) has an X-ray powder diffraction pattern (Cu K_{α} radiation) with at least five specific peaks at about 2-theta=7.5, 10.7, 12.8, 14.5 and 15.8°. In one embodiment, Form A of Compound (I) has an X-ray powder diffraction pattern with specific peaks at about 2-theta=7.5, 10.3, 10.7, 12.8, 14.5, 15.3, 15.8, 17.5, 19.6, and 21.3°. In one embodiment, Form A of Compound (I) has an X-ray powder diffraction pattern with at least five specific peaks at 2-theta=7.5, 10.7, 12.8, 14.5 and 15.8° wherein said values may be plus or minus 0.2° 2-theta. In one embodiment, Form A of Compound (I) has an X-ray powder diffraction pattern with specific peaks at 2-theta=7.5, 10.3, 10.7, 12.8, 14.5, 15.3, 15.8, 17.5, 19.6, and 21.3° wherein said values may be plus or minus 0.2° 2-theta. In one embodiment, Form A of Compound (I) has an X-ray powder diffraction pattern substantially the same as the X-ray powder diffraction pattern shown in FIG. 1.

The specific solid forms described herein provide X-ray powder diffraction patterns substantially the same as the X-ray powder diffraction patterns shown in the Figures and have the various 2-theta values as described herein. It will be understood that the 2-theta values of a X-ray powder diffraction pattern may vary slightly from one machine to another or from one sample to another, and so the values quoted are not to be construed as absolute.

It is known that an X-ray powder diffraction pattern may be obtained which has one or more measurement errors depending on measurement conditions (such as equipment or machine used). In particular, it is generally known that intensities in an X-ray powder diffraction pattern may fluctuate depending on measurement conditions. Therefore it should be understood that the solid forms described herein are not limited to the crystals that provide X-ray powder diffraction patterns that are identical to the X-ray powder diffraction pattern shown in the Figures, and any crystals providing X-ray powder diffraction patterns substantially the same as those shown in the Figures fall within the scope of the embodiments described herein. A person skilled in the art of X-ray powder diffraction is able to judge the substantial identity of X-ray powder diffraction patterns.

Persons skilled in the art of X-ray powder diffraction will realise that the relative intensity of peaks can be affected by, for example, grains above 30 μm in size and non-unitary aspect ratios, which may affect analysis of samples. The skilled person will also realise that the position of reflections can be affected by the precise height at which the sample sits in the diffractometer and the zero calibration of the diffractometer. The surface planarity of the sample may also have a small effect. Hence the diffraction pattern data presented are not to be taken as absolute values. (Jenkins, R & Snyder, R. L. ‘Introduction to X-Ray Powder Diffractometry’ John Wiley & Sons 1996; Bunn, C. W. (1948), Chemical Crystallography, Clarendon Press, London; Klug, H. P. & Alexander, L. E. (1974), X-Ray Diffraction Procedures). The peak intensities are described herein as vs (very strong), s (strong), m (medium) and w (weak) and correspond to % relative intensity (based on the most intense peak) of 25-100%, 10-25%, 3-10% and 1-3%, respectively. The relative intensities are derived from diffractograms measured with fixed slits.

Generally, a measurement error of a diffraction angle in an X-ray powder diffractogram is about 5% or less, in particular plus or minus 0.5° 2-theta, and such degree of a measurement error should be taken into account when considering the X-ray powder diffraction patterns in the Figures herein when reading the data described herein. Furthermore, it should be understood that intensities may fluctuate depending on experimental conditions and sample preparation (preferred orientation).

### Pharmaceutical Compositions Comprising Form G of Compound (I)

In an embodiment there is provided a pharmaceutical composition comprising Form G of Compound (I), as described herein, and a pharmaceutically acceptable excipient.

### Use of Solid Forms in Therapy

As a result of its modulation of RXFP1, Compound (I) is expected to be useful in therapy.

The term “therapy” is intended to have its normal meaning of dealing with a disease or condition in order to entirely or partially relieve one, some or all of its symptoms, or to correct or compensate for the underlying pathology. The term “therapy” also includes “prophylaxis” unless there are specific indications to the contrary. The terms “therapeutic” and “therapeutically” should be interpreted in a corresponding manner.

The term “prophylaxis” is intended to have its normal meaning and includes primary prophylaxis to prevent the development of the disease or condition and secondary prophylaxis whereby the disease or condition has already developed and the patient is temporarily or permanently protected against exacerbation or worsening of the disease or condition, or the development of new symptoms associated with the disease or condition.

The term “treatment” is used synonymously with “therapy”. Similarly the term “treat” can be regarded as “applying therapy” where “therapy” is as defined herein.

The term “therapeutically effective amount” refers to an amount of a compound which is effective to provide “therapy” in a subject, or to “treat” a disease or condition in a subject. The therapeutically effective amount may cause any of the changes observable or measurable in a subject as described in the definition of “therapy”, “treatment” and “prophylaxis” above. As recognized by those skilled in the art, effective amounts may vary depending on route of administration, excipient usage, and co-usage with other agents. For example, where a combination therapy is used, the amount of a pharmaceutically active agent(s) and the amount of the other pharmaceutically active agent(s) are, when combined, jointly effective to treat a targeted disorder or condition in the subject. In this context, the combined amounts are in a “therapeutically effective amount” if they are, when combined, sufficient to decrease the symptoms of a disease or condition responsive to modulation and/or agonism of RXFP1 as described above. Typically, such amounts may be determined by one skilled in the art.

As used herein, the terms “subject” and “patient” are used interchangeably. “Subjects” include, for example, mammals, for example, humans. In some embodiments, the subject is human.

Accordingly, the amorphous Compound (I), Form G of Compound (I), solid dispersions comprising amorphous Compound (I), or pharmaceutical compositions comprising Compound (I) described herein may be used in therapy, for example for treating a disease or disorder. Also provided is a method of treating a disease or disorder comprising administering to a subject or patient in need thereof a therapeutically effective amount of the amorphous Compound (I), Form G of Compound (I), solid dispersions comprising amorphous Compound (I), or pharmaceutical compositions comprising Compound (I) described herein.

It will be understood that the amorphous Compound (I), Form G of Compound (I), solid dispersions comprising amorphous Compound (I), or pharmaceutical compositions comprising Compound (I) described herein may be used in the treatment of cardiovascular diseases, for example for the treatment of heart failure, and hypertension.

As used herein, the term “heart failure” includes acute heart failure, chronic heart failure (CHF) and acute decompensated heart failure (ADHF). The term “heart failure” may also include more specific diagnoses such as heart failure with preserved ejection fraction (HFpEF), heart failure with mid-range ejection fraction (HFmrEF; also referred to as heart failure with mildly reduced ejection fraction), or heart failure with reduced ejection fraction (HFrEF).

As used herein, the term “resistant hypertension” is defined as the blood pressure of a hypertensive patient that remains elevated above goal despite the concurrent use of optimized doses of 3 antihypertensive agents of different classes, one of which is a diuretic, or a patient whose blood pressure is adequately controlled when receiving 4 or more antihypertensive medications concurrently (Carey et al., Hypertension, 2018, 72, e53-e90). The initial treatment of hypertension may be a calcium channel blocker (CCB), a blocker of the renin-angiotensin system (angiotensin-converting enzyme [ACE] inhibitor or angiotensin receptor blocker [ARB]), and a diuretic. For patients with rHT, further treatment may include a mineralocorticoid-receptor antagonist (MRA), a beta-blocker, and/or or a alpha-blocker. The subject with resistant hypertension may have a systolic blood pressure ≥140 mm Hg and/or diastolic blood pressure ≥90 mm Hg, typically when the subject is at rest. Alternatively, the subject with resistant hypertension may have a systolic blood pressure ≥130 mm Hg and/or diastolic blood pressure ≥80 mm Hg, typically when the subject is at rest. Alternatively, the subject with resistant hypertension may have a systolic blood pressure >150 mm Hg and/or diastolic blood pressure ≥90 mm Hg, typically when the subject is at rest. In some instances, the resistant hypertension may be resistant essential hypertension. Essential hypertension, also known as primary hypertension, is a form of hypertension with no known secondary cause identified.

The amorphous Compound (I), Form G of Compound (I), solid dispersions comprising amorphous Compound (I), or pharmaceutical compositions comprising Compound (I) described herein may also be used in the treatment of kidney disease (including chronic kidney disease), acute kidney injury, lung disease and fibrotic disorders, for example fibrotic disorders of the kidney, heart, lung and liver, wound healing (Sherwood O D (2004) Endocrine Reviews 25(2): 205-234), reversal of insulin resistance in diabetic patients (Bonner J S et al. (2013) Diabetes 62(9):3251-3260), the various forms of pulmonary hypertension, disorders that are a result of or a cause of arterial stiffness, reduced arterial elasticity, reduced arterial compliance and distensibility including hypertension, kidney disease, peripheral arterial disease, carotid and cerebrovascular disease (i.e. stroke and dementia), diabetes, microvascular disease resulting in end organ damage, coronary artery disease, and heart failure, and pre-eclampsia.

In one embodiment there is provided amorphous Compound (I) for use in therapy.

In one embodiment there is provided amorphous Compound (I) for use in the treatment of a disease or condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension. In one embodiment, the disease or condition is heart failure. In one embodiment, the disease or condition is resistant hypertension.

In one embodiment there is provided amorphous Compound (I) for use in the manufacture of a medicament for the treatment of a disease or condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension in a human patient. In one embodiment, the disease or condition is heart failure. In one embodiment, the disease or condition is resistant hypertension.

In one embodiment there is provided a method of treating a disease or condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension in a human patient in need of such treatment, comprising administering to the human patient a therapeutically effective amount of amorphous Compound (I). In one embodiment, the disease or condition is heart failure. In one embodiment, the disease or condition is resistant hypertension.

In one embodiment there is provided Form G of Compound (I) for use in the treatment of a disease or condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension. In one embodiment, the disease or condition is heart failure. In one embodiment, the disease or condition is resistant hypertension.

In one embodiment there is provided Form G of Compound (I) for use in the manufacture of a medicament for the treatment of a disease or condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension in a human patient. In one embodiment, the disease or condition is heart failure. In one embodiment, the disease or condition is resistant hypertension.

In one embodiment there is provided a method of treating a disease or condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension in a human patient in need of such treatment, comprising administering to the human patient a therapeutically effective amount of Form G of Compound (I). In one embodiment, the disease or condition is heart failure. In one embodiment, the disease or condition is resistant hypertension.

In one embodiment there is provided a solid dispersion comprising amorphous Compound (I) as described herein for use in the treatment of a disease or condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension. In one embodiment, the disease or condition is heart failure. In one embodiment, the disease or condition is resistant hypertension.

In one embodiment there is provided a solid dispersion comprising amorphous Compound (I) as described herein for use in the manufacture of a medicament for the treatment of a disease or condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension in a human patient. In one embodiment, the disease or condition is heart failure. In one embodiment, the disease or condition is resistant hypertension.

In one embodiment there is provided a method of treating a disease or condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension in a human patient in need of such treatment, comprising administering to the human patient a therapeutically effective amount of a solid dispersion comprising amorphous Compound (I) as described herein. In one embodiment, the disease or condition is heart failure. In one embodiment, the disease or condition is resistant hypertension.

In one embodiment there is provided a pharmaceutical composition comprising Compound (I) as described herein for use in the treatment of a disease or condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension. In one embodiment, the disease or condition is heart failure. In one embodiment, the disease or condition is resistant hypertension.

In one embodiment there is provided a pharmaceutical composition comprising Compound (I) as described herein for use in the manufacture of a medicament for the treatment of a disease or condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension in a human patient. In one embodiment, the disease or condition is heart failure. In one embodiment, the disease or condition is resistant hypertension.

In one embodiment there is provided a method of treating a disease or condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension in a human patient in need of such treatment, comprising administering to the human patient a therapeutically effective amount of a pharmaceutical composition comprising Compound (I) as described herein. In one embodiment, the disease or condition is heart failure. In one embodiment, the disease or condition is resistant hypertension.

### EXAMPLES

In general, all solvents used were commercially available and of analytical grade. Anhydrous solvents were routinely used for reactions. Phase Separators used in the examples are ISOLUTE® Phase Separator columns. The compounds described below were named using ChemDraw Professional version 19.0.0.22 from PerkinElmer.

Thermogravimetric Analysis (TGA) and Differential Scanning calorimetry (DSC) measurements were performed using a TG Discovery 550 (TA instruments, Germany) and DSC Discovery 2500 (TA instruments, Germany), respectively. 5.152 mg for TGA and 2.474 mg of the sample for DSC were weighed into an aluminum pan. The sample were then heated from room temperature to 350° C. for TGA and from 0° C. to 230° C. for DSC, with a heating rate of 10° C./min under a nitrogen purge of 100 mL/min. An empty aluminum pan was used as a reference for DSC.

The X-ray powder diffraction analysis was performed according to standard methods, which can be found in e.g. Kitaigorodsky, A. I. (1973), Molecular Crystals and Molecules, Academic Press, New York; Bunn, C. W. (1948), Chemical Crystallography, Clarendon Press, London; or Klug, H. P. & Alexander, L. E. (1974), X-ray Diffraction Procedures, John Wiley & Sons, New York.

### XRPD Method A

The X-ray powder diffraction (referred to herein as XRPD) pattern was determined by mounting a sample on a zero background holder single silicon crystal and spreading out the sample into a thin layer.

The powder X-ray diffraction was recorded with a theta-two theta scan axis and in one dimensional scan with Rigaku Miniflex 600 (wavelength of X-rays 1.5418 Å nickel-filtered Cu K_{α} radiation, 40 kV, 15 mA) equipped with D/Tex detector. Automatic variable divergence and anti scattering slits were used, and the samples were rotated at 80 revolution per minute during measurement. Samples were scanned using a 0.01° and 1°/min step width and scan speed respectively.

### XRPD Method B

Samples were mounted on a silicon wafer mount and analysed using the Bruker D4 Endeavour diffractometer (Cu anode, 2=1.5418 Å). Samples were measured in reflection geometry in θ-2θ configuration over the scan range 2° to 40° 2θ with a 0.12 second exposure per 0.02° increment (continuous scan mode). The X-rays were generated by a copper long-fine focus tube operated at 40 kV and 40 mA.

The following abbreviations were used

| | |
|---|---|
| Aq | Aq |
| B₂Pin₂ | 4,4,5,5-Tetramethyl-2-(4,4,5,5-tetramethyl- |
| | 1,3,2-dioxaborolan-2-yl)-1,3,2-dioxaborolane |
| Calcd | Calculated |
| DCM | Dichloromethane |
| DIA | Diisopropylamine |
| DIAD | Diisopropyl (E)-diazene-1,2-dicarboxylate |
| DIPEA | N-Ethyl-N-isopropyl-propan-2-amine |
| DMA | N,N-dimethylacetamide |
| DMSO | Dimethylsulfoxide |
| DPPA | Diphenylphosphoryl azide |
| DTBBPY | 4,4′-Di-tert-butyl-2,2′-dipyridyl |
| ESI | Electrospray ionization |
| Et | Ethyl |
| EtOAc | Ethylacetate |
| EtOH | Ethanol |
| h/hr | Hour(s) |
| HRMS | High resolution mass spectrometry |
| IPA | Isopropyl alcohol |
| IPAC | Isopropyl acetate |
| [Ir(COD)OMe]₂ | Bis(1,5-cyclooctadiene)di-μ-methoxydiiridium(I) |
| L | Litre |
| Me | Methyl |
| MeCN | Acetonitrile |
| mL | Millilitre |
| MeOH | Methanol |
| 2-Me—THF | 2-Methyltetrahydrofuran |
| Min | Minutes |
| MS | Mass spectrometry |
| MTBE | Methyl tert-butyl ether |
| NMR | Nuclear magnetic resonance |
| PE | Petroleum ether |
| Rt | Room temperature |
| Sat | Saturated |
| TEA | Triethylamine |
| TFA | Trifluoroacetic acid |
| THF | Tetrahydrofuran |
| TLC | Thin layer chromatography |
| UHPLC | Ultra High Performance Liquid Chromatography |

### Preparation of Compound (I)

### Intermediate 1: Ethyl 8-methyl-1,4-dioxaspiro[4.5]decane-8-carboxylate

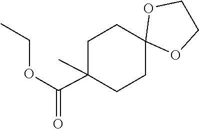 [SMILES: CCOC(=O)C1(C)CCC2(CC1)OCCO2]

A solution of DIA (576 mL, 413 g, 4.08 mol) in THF (3.50 L) was cooled to −50 to −40° C. and a solution of n-BuLi (2.5 M in hexane, 1.09 kg, 3.92 mol) was added over 3 h, maintaining the temperature between −50 to −40° C. The solution was stirred for 3 h at −50 to −40° C., followed by the addition of a solution of ethyl 1,4-dioxaspiro[4.5]decane-8-carboxylate (700 g, 3.27 mol, 2.34 M in THF) over 2 h, maintaining the temperature between −50 to −40° C. The reaction mixture was stirred for 4 h at −50 to −40° C. before the addition of methyl iodide (603 g, 4.25 mol, 3.04 M in THF) over 3 h, maintaining the temperature between −50 to −30° C. The reaction mixture was further stirred for 2 h at −50 to −30° C. followed by the addition of aq NH₄Cl (3.50 L, 20% w/w in H₂O) over 1 h, maintaining the temperature <0° C. The solution was warmed to between 15 to 25° C., held for 0.5 h then the layers were separated and the organic layer washed with aq NH₄Cl (2×3.50 L, 20% w/w in H₂O). Exchange of the organic reaction solvent from THF to EtOH under reduced pressure, maintaining the temperature <45° C., gave the title compound as a 27% w/w solution in EtOH (2.51 kg, 2.91 mol, 89%). ¹H NMR for purified compound (400 MHz CDCl₃) δ 1.18 (3H, s), 1.27-1.22 (3H, m), 1.47-1.35 (2H, m), 1.70-1.56 (4H, m), 2.13 (2H, d), 3.92 (4H, s), 4.14 (2H, q), MS (ESI): m/z [M+H]⁺ 229.2.

### Intermediate 2:8-Methyl-1,4-dioxaspiro[4.5]decane-8-carboxylic acid

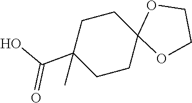

To a solution of Intermediate 1 (1.13 kg, 1.31 mol, 27% w/w in EtOH) was added EtOH (900 mL) followed by aq NaOH (2.63 L, 5.26 mol, 2 M in H₂O) maintaining the temperature between 15 to 30° C. The solution was heated to between 50 to 60° C., then held for 6 h before cooling to 15 to 30° C. and concentration of the solution to between 1.8 to 2.4 L under reduced pressure. Hexane (1.50 L) was added and the layers separated. The aq layer was collected and the pH adjusted to between 3 to 4 by the addition of aq HCl (1.30 L, 5.2 mol, 4 M in H₂O) maintaining the temperature <20° C. This aq solution was extracted with DCM (2×1.50 L) and the combined organic phases were concentrated under reduced pressure, maintaining the temperature <30° C. to give the title compound as a 17% w/w solution in DCM (1.43 kg, 1.24 mol, 94%). ¹H NMR for purified compound (500 MHz CDCl₃) δ 1.25 (3H, s), 1.53 (2H, dt), 1.62-1.72 (4H, m), 2.05-2.19 (2H, m), 3.93 (4H, s). MS (ESI): m/z [M+Na]⁺ 223.1.

### Intermediate 3:1-Methyl-4-oxocyclohexane-1-carboxylic acid

### Method A

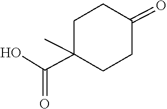

To a solution of Intermediate 2 (367 g, 250 mmol, 14% w/w in DCM) was added TFA (95.3 mL, 142 g, 1.25 mol). The reaction temperature was maintained between 25 to 35° C. for 20 h before cooling to between 0 to 10° C. H₂O (250 mL) was added to the reaction solution and the pH of the aq phase adjusted to between 9 and 10 by the addition of aq NaOH (440 mL, 1.76 mol, 4 M in H₂O). The layers were separated and the aq layer was retained and cooled to between 0 to 10° C. The pH was adjusted to between 2 and 3 by the addition of aq HCl (73.5 mL, 294 mmol, 4 M in H₂O) then extracted with DCM (3×250 mL) and the combined DCM solutions concentrated to between 150 to 200 mL under reduced pressure. Exchange of the organic reaction solvent from DCM to MeCN under reduced pressure, maintaining the temperature <40° C., gave the title compound as a 30% w/w solution in MeCN (119 g, 227 mmol, 91%). ¹H NMR for purified compound (400 MHz CDCl₃) δ 1.39 (3H, s), 1.73 (2H, td), 2.43 (6H, m). MS (ESI): m/z [M+H]⁺ 157.1.

### Method B

To a solution of Intermediate 2 (6.17 kg, 3.83 mol, 12.4% in DCM) was added TFA (1.42 L, 2.18 kg, 19.13 mol). The reaction temperature was maintained between 25 to 35° C. for 20 h before cooling to between 0 to 10° C. A solution of aq NaOH (918 g, 22.96 mol dissolved in 7.66 L H₂O) was added to the reaction solution and the pH of the aqueous phase was adjusted to between 9 and 11. The layers were separated and the aq layer was retained and cooled to between 0 to 10° C. Addition of DCM (3.83 L) followed by aq HCl (1.52 L, 6.08 mol, 4 M in H₂O) adjusts the pH to between 3 and 4. The organic layer was retained and the aqueous extracted with DCM (2×3.83 L) and the combined organic phase was washed with brine (2.3 L, 15% w/w NaCl). The organic phase was concentrated under reduced pressure to 2.3 to 3.1 L. Exchange of the organic reaction solvent from DCM to MeCN under reduced pressure, maintaining the temperature <45° C., gave the title compound as a 18% solution in MeCN (2.85 kg, 3.32 mol, 87%). ¹H NMR for purified compound (400 MHz CDCl₃) δ 1.39 (3H, s), 1.73 (2H, td), 2.43 (6H, m). MS (ESI): m/z [M+H]⁺ 157.1.

### Intermediate 4: Naphthalen-1-ylmethyl 1-methyl-4-oxocyclohexane-1-carboxylate

### Method A

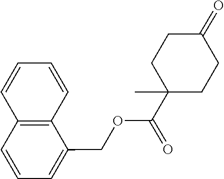 [SMILES: CC1(C(=O)OCc2cccc3ccccc23)CCC(=O)CC1]

To a solution of Intermediate 3 (119 g, 192 mmol, 25% w/w in MeCN) was added 1-chloromethylnaphthalene (32.2 g, 183 mmol) followed by DIPEA (70.0 mL, 49.7 g, 384 mmol) and NaI (2.88 g, 19.2 mmol). The solution was heated to between 50 to 60° C. for 8 h before cooling to between 0 to 10° C. H₂O (240 mL) was added and the pH of the reaction mixture adjusted to between 3 and 4 by the addition of aq HCl (55.0 mL, 220 mmol, 4 M in H₂O). The reaction mixture was extracted with MTBE (2×150 mL) and the combined organic phases washed with aq NaHCO₃ (150 mL, 144 mmol, 8% w/w in H₂O). The organic reaction solvent was exchanged from MTBE to IPA under reduced pressure, maintaining the temperature <40° C. The temperature of the reaction solution was lowered to between −10 to 3° C. and the solution stirred for 2 h, upon which a solid precipitate formed. The solids were filtered and dried under N₂ for 15 h to give the title compound as a white solid (42.8 g, 144 mmol, 74%); ¹H NMR (500 MHz, CDCl₃) 1.30 (3H, s), 1.65 (2H, td), 2.16-2.47 (6H, m), 5.66 (2H, s), 7.46 (1H, dd), 7.51-7.63 (3H, m), 7.78-7.93 (2H, m), 7.93-8.05 (1H, m). MS (ESI): m/z [M+Na]⁺ 319.1.

### Method B

To a solution of Intermediate 3 (2.66 kg, 3.09 mol, 18.2% in MeCN) was added 1-chloromethylnapthalene (535 g, 2.94 mol) followed by potassium carbonate (513 g, 3.71 mol) and a further portion of fresh MeCN (714 mL). The suspension was heated to between 50 to 60° C. for 17 h before cooling to 25 to 30° C. The solid was removed by filtration through a Celite pad, which was washed through with MeCN (2×967 mL). Concentrate the filtrates to 1.45 to 1.93 L under reduced pressure. The MeCN was exchanged to isopropanol under reduced pressure, maintaining the temperature <50° C. The temperature of the mixture was lowered to between 20 to 25° C., upon which a solid precipitate formed. The mixture was cooled further to −10 to 0° C., and the solids were then filtered, washed with isopropanol and dried under N₂ to give the title compound as a white solid (752.6 g, 2.49 mol, 80.5%); ¹H NMR (500 MHz, CDCl₃) 1.30 (3H, s), 1.65 (2H, td), 2.16-2.47 (6H, m), 5.66 (2H, s), 7.46 (1H, dd), 7.51-7.63 (3H, m), 7.78-7.93 (2H, m), 7.93-8.05 (1H, m). MS (ESI): m/z [M+Na]⁺ 319.1.

### Intermediate 5: Methyl 5-(1,3,6,2-dioxazaborocan-2-yl)-4-fluoro-2-methoxybenzoate

### Method A

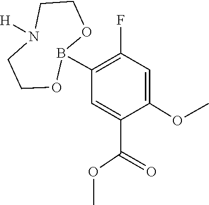 [SMILES: COC(=O)c1cc(B2OCCNCCO2)c(F)cc1OC]

B₂Pin₂ (362 g, 1.43 mol) was added to 2-Me-THF (1.75 L) that had been degassed with N₂ to <1% oxygen. The solution was held between 20 to 30° C. and methyl 4-fluoro-2-methoxybenzoate was added (250 g, 1.36 mol). DTBBPY (1.09 g, 4.10 mmol) was added and the reaction vessel evacuated and re-filled with N₂ until the oxygen level was <0.5%. [Ir(COD)OMe]₂ (1.35 g, 2.04 mmol) was added and the reaction vessel evacuated and re-filled with N₂ until the oxygen level was <0.5%. The reaction mixture was heated to between 80 to 85° C. and held at that temperature for a further 2 h. The reaction mixture was cooled to between 0 to 5° C. followed by the slow addition of diethanolamine (428 g, 4.07 mol, 10.9 M in IPA) over a period of 2.5 h, with the concurrent generation of H₂ gas. The reaction mixture was stirred for 2.5 h between 0 to 5° C., followed by filtration and washing of the solids with 2-Me-THF (3×750 mL). The solid was dried under N₂ for 10 h to give the title compound as a white solid (356 g, 1.20 mol, 88%); ¹H NMR (500 MHz, DMSO-d6) δ 2.81-2.89 (2H, m), 3.14 (2H, dq), 3.71 (2H, ddd), 3.74 (3H, s), 3.78 (3H, s), 3.84 (2H, td), 6.77 (1H, d), 7.10 (1H, s), 7.83 (1H, d). MS (ESI): m/z [M+H]⁺ 297.1.

### Method B

B₂Pin₂ (29.0 g, 114 mmol) and methyl 4-fluoro-2-methoxybenzoate (20.6 g, 109 mmol) were added to 2-Me-THF (140 mL) that had been degassed with N₂ to <1% oxygen. The solution was held between 20 to 30° C. then DTBBPY (88 mg, 0.33 mmol) and [Ir(COD)OMe]₂ (108 mg, 0.16 mmol) were added and the reaction vessel evacuated and re-filled with N₂ until the oxygen level was <0.5%. The reaction mixture was heated to between 80 to 85° C. and held at that temperature for a further 3 h. The reaction mixture was cooled to between 0 to 10° C. followed by the slow addition of isopropanol (12.4 mL, 218 mmol), with the concurrent generation of H₂ gas. Addition of seed (100 mg of Intermediate 5) followed by addition of diethanolamine (22.84 g, 218 mmol) dissolved in IPA (20 mL) gave a mobile slurry. The slurry was warmed to 20 to 30° C. and the solid collect by filtration. It was then washed with 2-Me-THF (160 ml) and the solid was dried under N₂ for 10 h to give the title compound as a white solid (29.1 g, 96 mol, 88%); ¹H NMR (500 MHz, DMSO-d6) δ 2.81-2.89 (2H, m), 3.14 (2H, dq), 3.71 (2H, ddd), 3.74 (3H, s), 3.78 (3H, s), 3.84 (2H, td), 6.77 (1H, d), 7.10 (1H, s), 7.83 (1H, d). MS (ESI): m/z [M+H]⁺ 297.1.

### Intermediate 6: Methyl 4-fluoro-5-hydroxy-2-methoxybenzoate

### Method A

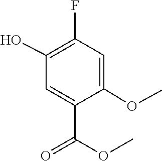

To a suspension of Intermediate 5 (350 g, 1.18 mol) in H₂O (1.05 L) was added THF (1.75 L) and the reaction mixture stirred until a clear solution is obtained. (NH₄)₂CO₃ (136 g, 1.41 mol) was added and the heterogenous mixture cooled to between 0 to 10° C. NaBO₃·4H₂O (217 g, 1.41 mol) was added in 10 equal portions over a period of 2 h maintaining the reaction temperature between 0 to 30° C. The reaction temperature was adjusted to between 20 to 30° C. and held for 1 h. An aq solution of NaHSO₃ (1.96 L, 942 mmol, 0.48 M in H₂O) was added over 3 h and the reaction mixture stirred for an additional 0.5 h. The reaction mixture was filtered, the solids washed with ethylacetate (700 mL) and the filtrate and wash combined to give a biphasic solution. The solution was separated and the retained organic phase solvent exchanged from THF/ethylacetate to MeOH under reduced pressure, maintaining the temperature <40° C. H₂O (3.50 L) was added drop-wise over a period of 4 h and the reaction mixture cooled to between 0 to 5° C. and held for 2 h. The reaction mixture was filtered, the collected solids washed with H₂O (3×350 mL) and dried under hot air at <40° C. to give the title compound as a white solid (195 g, 974 mmol, 83% yield); ¹H NMR (500 MHz, CDCl₃) § 3.82 (3H, s), 3.86 (3H, s), 6.72 (1H, d), 7.54 (1H, d). MS (ESI): m/z [M+H]⁺ 201.0.

### Method B

Intermediate 5 (32.41 g, 67.3 mmol) was dissolved in 2-Me-THF (100 mL) with acetic acid (12.13 g, 202 mmol) and cooled to between 0 to 10° C. Hydrogen peroxide solution (30% w/w, 9.16 g, 80.8 mmol) was added over 2 hours and then the reaction temperature was adjusted to between 20 to 30° C. and held for 18 hours. An aq solution of Na₂S₂O₃·5H₂O (20% w/w, 50 mL) quenches the mixture and gives a phase separation. The aqueous is discarded, and the organic washed twice with aq solution of Na₂S₂O₃·5H₂O (5% w/w, 100 mL). The organic phase was concentrated to 60 mL under reduced pressure followed by another 2 vacuum distillations with 2-Me-THF (100 mL) to give a dissolved solution at 35 to 45° C. Nucleation was controlled by addition of seed (100 mg of Intermediate 6) followed by slow addition of 300 mL n-heptane over 5 hours. The resulting slurry was adjusted to between 20 to 30° C. and stirred overnight prior to filtration. The collected solid was washed with n-heptane (2×60 mL) and dried to give the title compound as a white solid (12.5 g, 62.5 mmol, 93% yield); ¹H NMR (500 MHz, CDCl₃) δ 3.82 (3H, s), 3.86 (3H, s), 6.72 (1H, d), 7.54 (1H, d). MS (ESI): m/z [M+H]⁺ 201.0.

### Intermediate 7: (1R,2R,3S,4S)-3-(Methoxycarbonyl)bicyclo[2.2.1]hept-5-ene-2-carboxylic acid

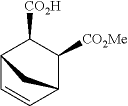 [SMILES: COC(=O)[C@@H]1[C@H](C(=O)O)[C@H]2C=C[C@@H]1C2]

To a solution of (3aR,4R,7S,7aS)-3a,4,7,7a-tetrahydro-4,7-methanoisobenzofuran-1,3-dione (387 g, 2.36 mol) in toluene (4.64 L) was added quinidine (843 g, 2.60 mol) followed by toluene (774 mL). The reaction mixture was cooled to between −10 to −5° C. and MeOH (227 g, 286 mL, 7.08 mol) was added drop-wise over 1.5 h before holding at between −10 to −5° C. for 14 h. The reaction mixture was warmed to between −5 to 5° C., held for 2 h, then filtered. The solids were washed with toluene (3×387 mL), the filtrate and washes combined and cooled to between 0 to 10° C. In a separate vessel an aq solution of HCl (590 mL, 7.08 mol, 12 M in H₂O) and NaCl (1.24 kg, 21.2 mol) were added to H₂O (6.39 L) and the resulting solution added dropwise to the main reaction vessel, maintaining the reaction solution <10° C. The reaction mixture was warmed to between 10 to 20° C., held for 0.5 h then filtered. The solids were washed with toluene (1.94 L), the filtrate and wash combined, and the biphasic solution separated. The organic phase was washed with aq NaCl (3.87 L, 20% w/w in H₂O) and stored at <5° C. to give the title compound as a 5.9% w/w solution in toluene (6.19 kg, 1.83 mol, 78%); ¹H NMR for purified compound (400 MHz DMSO-d₆) δ 1.25-1.32 (1H, m), 1.95 (1H, d), 2.48-2.50 (2H, m), 2.93 (2H, s), 3.51 (3H, s), 6.15-6.22 (2H, m), 12.21 (1H, s). MS (ESI): m/z [M+Na]⁺ 219.1.

### Intermediate 8: Methyl (1S,2S,3R,4R)-3-aminobicyclo[2.2.1]hept-5-ene-2-carboxylate hydrochloride

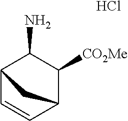 [SMILES: Cl ; COC(=O)[C@@H]1[C@H](N)[C@H]2C=C[C@@H]1C2]

To a solution of Intermediate 7 in toluene (6.19 kg, 5.9% w/w, 1.85 mol) at between −5 to 5° C. was added TEA (307 mL, 223 g, 2.22 mol) followed by DPPA (538 g, 1.94 mol), maintaining the reaction solution <5° C. The reaction mixture was stirred for 4 h at between −5 to 5° C. then TEA was added (767 mL, 557 g, 5.55 mol) followed by citric acid (352 g, 1.85 mol). The reaction mixture was stirred for 6 h at between −5 to 5° C. then H₂O (3.6 L) was added maintaining the reaction solution <10° C. The biphasic reaction solution was stirred for 0.5 h, the phases separated and the organic phase washed with H₂O (3.6 L) and aq NaCl (3.6 L, 15% w/w in H₂O) then stored between 2 to 8° C. to give methyl (1S,2S,3R,4R)-3-(azidocarbonyl)bicyclo[2.2.1]hept-5-ene-2-carboxylate (Intermediate 9) as a solution in toluene that was used directly in the next step. Intermediate 9 as a solution in toluene at between 2 to 8° C. was added over 2 h to a reactor containing toluene (1.80 L) at between 70 to 80° C., maintaining the reaction temperature <80° C. The resulting solution was stirred for 1 h before cooling to between 20 to 30° C. Exchange of the organic reaction solvent from toluene to 1,4-dioxane under reduced pressure, maintaining the temperature <50° C., gave Methyl (1S,2S,3R,4R)-3-isocyanatobicyclo[2.2.1]hept-5-ene-2-carboxylate (Intermediate 10) as a solution in 1,4-dioxane that was used directly in the next step. To a solution of Intermediate 10 in 1,4-dioxane at between 10 to 20° C. was added HCl (420 mL, 1.68 mol, 4 M in 1,4-dioxane) followed by H₂O (360 mL, 1.68 mol, 4.67 M in 1,4-dioxane). The reaction mixture was warmed to between 25 to 35° C. and held for 16 h. MTBE (1.65 L) was added drop-wise and the reaction mixture filtered, the solids washed with MTBE/1,4-dioxane (1:1, 660 mL) and MTBE (660 mL), then dried at between 30 to 40° C. under vacuum to give the title compound as a white solid (258 g, 1.27 mol, >99% ee, 75%); ¹H NMR (400 MHz, DMSO-d6) δ 1.45 (1H, d), 2.04 (1H, d), 2.52-2.67 (1H, m), 2.94-3.10 (2H, m), 3.19 (1H, d), 3.65 (3H, s), 6.21 (1H, m), 6.30 (1H, m), 8.34 (3H, s). MS (ESI): m/z [M+H]⁺ 168.1.

### Intermediate 11: Naphthalen-1-ylmethyl (1r,4r)-4-hydroxy-1-methylcyclohexane-1-carboxylate

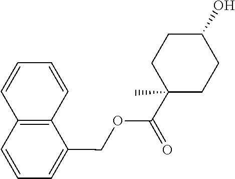 [SMILES: C[C@]1(C(=O)OCc2cccc3ccccc23)CC[C@H](O)CC1]

### ROUTE A

To a solution of Na₂HPO₄·12H₂O (8.25 g, 23.0 mmol), NaH₂PO₄ (0.55 g, 4.48 mmol) and MgCl₂ (0.11 g, 1.10 mmol) in H₂O (550 mL) at 20 to 30° C. was added Intermediate 4 (50.0 g, 169 mmol) as a solution in IPA (450 mL). The pH of the reaction solution was adjusted to between 7.3 to 7.8 using 6 M HCl and NAD+ (0.66 g, 1.00 mmol) was added followed by ADH-230 (7.50 g, 0.15 wt %). ADH-230 is an alcohol dehydrogenase available from Johnson Matthey PLC, UK (catalogue no. ADH-230). The reaction mixture was then held at 33 to 37° C. for 18 h before concentration to between 300 and 400 mL under reduced pressure, maintaining the temperature <45° C. NaCl (150 g), Celite® (20.0 g, 0.4 wt %) and MTBE (500 mL) was added and the reaction held for 0.5 h. The mixture was filtered and the filter cake washed with MTBE (250 mL). The combined filtrate was separated and the aq phase extracted with MTBE (500 mL). The organic phases were combined and washed with H₂O (250 mL) before solvent exchange to THF under reduced pressure, maintaining the temperature <45° C., gave the title compound (138 g, 33% w/w %, >99:1 trans: cis, <0.1% IPA, 92% yield) as a solution in THF that was used directly in the next step. ¹H NMR for purified compound (500 MHz, CDCl₃) δ 1.21 (3H, s), 1.48-1.58 (2H, m), 1.62-1.77 (4H, m), 1.82-1.93 (2H, m), 3.74-3.77 (1H, m), 5.57 (2H, s), 7.41-7.48 (1H, m), 7.48-7.57 (3H, m), 7.85 (1H, d), 7.87-7.91 (1H, m), 7.98 (1H, d). MS (ESI): m/z [M+Na]⁺ 321.1.

### ROUTE B

A solution of lithium tri-sec-butylborohydride (1.06 g, 5.6 mmol) in THF (5 mL) was added dropwise to a stirred solution of Intermediate 4 (1.00 g, 3.37 mmol) in THF (10 mL) cooled to −78° C., over a period of 1 min under nitrogen. The resulting solution was stirred at −78° C. for 2 h. The reaction mixture was quenched with 0.1 M HCl (10 mL) at −78° C. and then extracted with EtO Ac (3×50 mL). The organic layers were pooled and dried over Na₂SO₄ filtered and evaporated. The residue was purified by preparative TLC (EtOAc/PE, 1:3), to afford the title compound (0.488 g, 48.5%) as a pale yellow gum. The isolated material had a 3:100 cis/trans ratio. ¹H NMR (400 MHz, CDCl₃) δ 1.21-1.25 (s, 3H), 1.37-1.49 (m, 1H), 1.49-1.61 (m, 2H), 1.61-1.74 (m, 4H), 1.83-1.95 (m, 2H), 3.74-3.83 (dq, 1H), 5.57-5.61 (s, 2H), 7.43-7.54 (dd, 1H), 7.50-7.61 (m, 3H), 7.84-7.94 (m, 2H), 7.97-8.04 (m, 1H).). MS (ESI): m/z [M+Na]⁺ 321.

### Intermediate 12: Methyl 4-fluoro-2-methoxy-5-(((1s,4s)-4-methyl-4-((naphthalen-1-ylmethoxy)carbonyl)cyclohexyl)oxy)benzoate

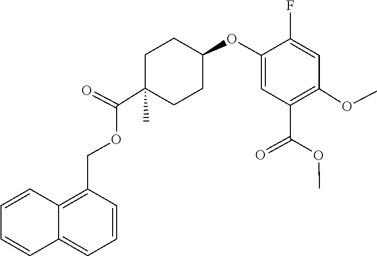 [SMILES: COC(=O)c1cc(O[C@H]2CC[C@@](C)(C(=O)OCc3cccc4ccccc34)CC2)c(F)cc1OC]

To a solution of Intermediate 11 in THF (736 g, 34% w/w, 839 mmol) was added THF (156 mL), PPh₃ (248 g, 944 mmol) and Intermediate 6 (140 g, 699 mmol). The solution was heated to 30° C. prior to the drop-wise addition of DIAD (184 g, 909 mmol) over 1 h maintaining the reaction temperature <40° C. The solution was held at between 3° and 40° C. for 1 h before cooling to between 2° and 30° C. followed by the addition of an aq solution of NaCl (700 mL, 20% w/w in H₂O). The layers were separated and the crude solution of the title compound in THF was used directly in the next step. ¹H NMR for purified compound (500 MHz, CDCl₃) δ 1.17 (3H, s), 1.20-1.30 (2H, m), 1.58 (2H, qd), 1.88-1.98 (2H, m), 2.29 (2H, d), 3.84 (3H, s), 3.88 (3H, s), 4.05 (1H, tq), 5.61 (2H, s), 6.72 (1H, d), 7.43-7.58 (5H, m), 7.82-7.94 (2H, m), 8.00 (1H, d). MS (ESI): m/z [M+Na]⁺ 503.2.

### Intermediate 13:4-Fluoro-2-methoxy-5-(((1s,4s)-4-methyl-4-((naphthalen-1-ylmethoxy)carbonyl)cyclohexyl)oxy)benzoic acid

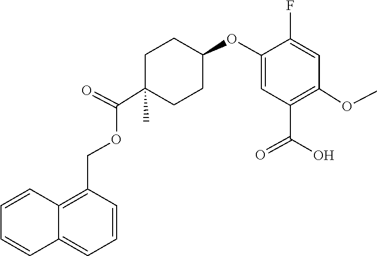 [SMILES: COc1cc(F)c(O[C@H]2CC[C@@](C)(C(=O)OCc3cccc4ccccc34)CC2)cc1C(=O)O]

To the crude solution of Intermediate 12 used directly from the previous step at between 0 and 5° C., was added a aq solution of LiOH·2H₂O (88.0 g, 2.10 mol, in 525 mL of H₂O) over 1 h maintaining the reaction temperature <10° C. The solution was warmed to between 15 and 30° C. and vigorously stirred for 16 h. IPAC (1.68 L) was added and the solution cooled to between 0 and 10° C. followed by the drop-wise addition of H₃PO₄ (1.26 L, 2.52 M, 2 M in H₂O), maintaining the reaction temperature <10° C., to give a solution pH of between 4.0 and 5.0. The organic layer was separated and washed with of an aq solution of NaCl (700 mL, 20% w/w in H₂O). The THF was removed under reduced pressure, maintaining the temperature <50° C. and IPAC (4.20 L) was added to give the title compound in IPAC that was used directly in the next step. ¹H NMR for purified compound (500 MHz, CDCl₃) δ 1.18 (3H, s), 1.22-1.36 (2H, m), 1.58 (2H, qd), 1.95 (2H, dt), 2.29 (2H, d), 4.02 (3H, s), 4.19 (1H, td), 5.60 (2H, s), 6.82 (1H, d), 7.46 (1H, dd), 7.49-7.62 (3H, m), 7.78 (1H, d), 7.82-7.94 (2H, m), 7.99 (1H, d).

### Intermediate 14:4-Fluoro-2-methoxy-5-(((1s,4s)-4-methyl-4-((naphthalen-1-ylmethoxy)carbonyl)cyclohexyl)oxy)benzoate cyclohexanaminium salt

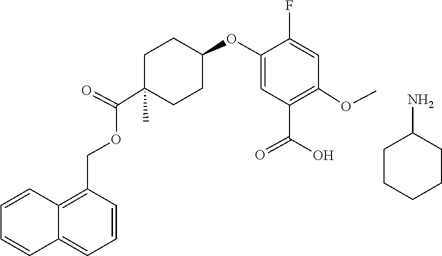

To a crude solution of Intermediate 13 in IPAC used directly from the previous step at between 5° and 55° C., was added a solution of cyclohexylamine (280 mL, 699 mmol, 2.5 M in IPAC) drop-wise over 3 h. The heterogenous slurry was stirred at between 5° and 55° C. for 0.5 h then at between 4° and 45° C. for a further 1 h. The reaction mixture was filtered and the solids washed with IPAC (3×0.98 L) pre warmed to between 4° and 45° C. and dried under a flow of N₂ at 45° C. for 16 h. To the dried collected solids was added MeOH (3.64 L) and the mixture heated to between 55 and 56° C. H₂O (1.58 L) was added drop-wise over 1 h then the mixture stirred for 1 h before cooling to between 0 and 5° C. over 3 h. The heterogenous slurry was held for a further 1 h then filtered, washed with 5:3 MeOH: H₂O at 0° C. (2×750 mL) and the solids dried under N₂ at 45° C. for 16 h to give the title compound as a white solid (332 g, 85% from methyl 4-fluoro-5-hydroxy-2-methoxybenzoate); ¹H NMR (500 MHz, CDCl₃) δ 0.96 (1H, ddt), 1.03-1.36 (6H, m), overlapping 1.14 (3H, S), 1.46-1.7 (5H, m), 1.91 (4H, dt), 2.26 (2H, d), 2.81 (1H, t), 3.76 (3H, s), 4.03 (1H, tt), 5.59 (2H, s), 6.65 (1H, d), 7.37-7.49 (2H, m), 7.49-7.6 (3H, m), 7.81-7.93 (2H, m), 7.98 (1H, d). MS (ESI): m/z [M+Na]⁺ 489.2.

### Intermediate 15: Methyl (1S,2S,3R,4R)-3-(4-fluoro-2-methoxy-5-(((1s,4S)-4-methyl-4-((naphthalen-1-ylmethoxy)carbonyl)cyclohexyl)oxy)benzamido)bicyclo[2.2.1]hept-5-ene-2-carboxylate

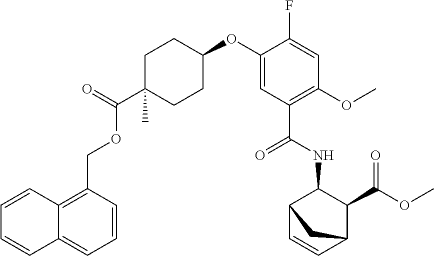 [SMILES: COC(=O)[C@@H]1[C@H](NC(=O)c2cc(O[C@H]3CC[C@@](C)(C(=O)OCc4cccc5ccccc45)CC3)c(F)cc2OC)[C@H]2C=C[C@@H]1C2]

To a solution of Intermediate 14 (149 g, 264 mmol) in DCM (750 mL) at between 15 and 30° C. was added H₂O (450 mL) followed by the slow addition of HCL (300 mL, 1 M in H₂O). The biphasic solution was stirred for 0.5 h then separated and the organic phased washed with HCl (750 mL, 0.2 M in H₂O) then with H₂O (3×750 mL). The organic solution was concentrated under reduced pressure, maintaining the temperature below 30° C., to dry to <0.1% H₂O. The solution was diluted with DCM (450 mL) to bring the total volume to 750 mL before the addition of Intermediate 8 (59.3 g, 291 mmol) to give a heterogenous slurry. To this mixture was added DIPEA (137 g, 1.06 mol) followed by T3P (252 g, 397 mmol, 50% w/w in EtAOc) and the solution stirred for 1 h. The solution was cooled to between 0 and 10° C. followed by the addition of H₂O (750 mL) and subsequently stirred for a further 0.5 h. The biphasic solution was separated and the organic phase washed with H₂O (2×750 mL) before solvent exchange to THF under reduced pressure gave the title compound in THF that was used directly in the next step. ¹H NMR for purified compound (500 MHz, CDCl₃) δ 1.16 (3H, s), 1.25 (2H, td), 1.49-1.69 (3H, m), 1.92-2.01 (2H, m), 2.04-2.1 (1H, m), 2.28 (2H, d), 2.71 (1H, dd), 2.83 (1H, s), 2.92-3.05 (1H, m), 3.61 (3H, s), 3.93 (3H, s), 4.17 (1H, td), 4.46 (1H, td), 5.60 (2H, s), 6.25 (2H, ddd), 6.72 (1H, d), 7.46 (1H, dd), 7.48-7.6 (3H, m), 7.81-7.95 (3H, m), 7.99 (1H, d), 8.60 (1H, d). MS (ESI): m/z [M+H]⁺ 616.3.

### Intermediate 16: (1S,2S,3R,4R)-3-(4-Fluoro-2-methoxy-5-(((1s,4S)-4-methyl-4-((naphthalen-1-ylmethoxy)carbonyl)cyclohexyl)oxy)benzamido)bicyclo[2.2.1]hept-5-ene-2-carboxylic acid

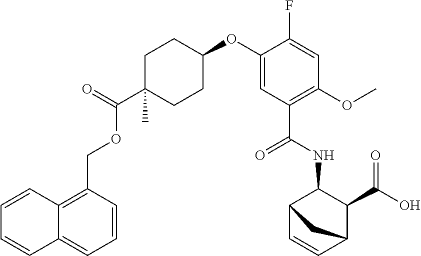 [SMILES: COc1cc(F)c(O[C@H]2CC[C@@](C)(C(=O)OCc3cccc4ccccc34)CC2)cc1C(=O)N[C@H]1[C@@H](C(=O)O)[C@@H]2C=C[C@H]1C2]

A crude solution of Intermediate 15 in THF (750 mL) from the previous step was cooled to between 0 and 5° C. An aq solution of LiOH·2H₂O (27.7 g, 661 mmol, in 150 mL of H₂O) was added and the solution held for 36 h. The pH of the solution was adjusted to 2 with the portion wise slow addition of HCl (0.5 M, 1.45 L, 2.90 mol) and held for 1 h between 0 and 5° C. The heterogenous slurry was filtered and the solids washed with 1:3 MeOH: H₂O at 0° C. (600 mL) and the solids dried under N₂ at 45° C. for 16 h to give crude title compound as a white solid (158 g, 99%). The crude (150 g) was slurried in IPAC (1.13 L) at between 6° and 65° C. for 0.5 h. The heterogenous mixture was cooled to between 0 and 5° C. over 3 h then further stirred for 1 h before filtration. The collected solids were with IPAC at between 0 and 5° C. (2×300 mL) then dried under N₂ at 45° C. for 12 h to give the title compound as a white solid (127 g, 82% from Intermediate 14); ¹H NMR (500 MHz, CDCl₃) δ 1.16 (3H, s), 1.2-1.35 (2H, m), 1.50-1.69 (3H, m), 1.89-2.08 (3H, m), 2.27 (2H, ddd), 2.72 (1H, dd), 2.80 (1H, s), 3.06 (1H, s), 3.75 (3H, s), 4.15 (1H, tt), 4.43-4.54 (1H, m), 5.59 (2H, s), 6.24 (2H, ddd), 6.53 (1H, d), 7.45 (1H, dd), 7.47-7.58 (3H, m), 7.8-7.9 (3H, m), 7.94-8.05 (1H, m), 8.59 (1H, d). MS (ESI): m/z [M+H]⁺ 602.3.

### Intermediate 17: Naphthalen-1-ylmethyl (1S,4s)-4-(2-fluoro-4-methoxy-5-(((1R,2R,3S,4S)-3-(((1-methylcyclobutyl)methyl)carbamoyl)bicyclo[2.2.1]hept-5-en-2-yl)carbamoyl)phenoxy)-1-methylcyclohexane-1-carboxylate

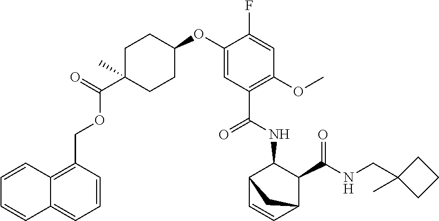 [SMILES: COc1cc(F)c(O[C@H]2CC[C@@](C)(C(=O)OCc3cccc4ccccc34)CC2)cc1C(=O)N[C@H]1[C@@H](C(=O)NCC2(C)CCC2)[C@@H]2C=C[C@H]1C2]

To a solution of DIPEA (6.45 g, 49.9 mmol) in DCM (300 mL) at between 0 and 5° C. was added Intermediate 16 (30.6 g, 49.9 mmol) followed by (1-methylcyclobutyl) methanamine hydrochloride (8.63 g, 62.4 mmol). DIPEA (25.8 g, 200 mmol) was added drop-wise maintaining the temperature between 0 and 5° C., followed by the addition of T3P (50.8 g, 79.8 mmol, 50% w/w in EtAOc) over 0.5 h. The solution was warmed to between 15 and 25° C. and stirred for 1 h followed by the drop-wise addition of H₂O (150 mL) maintaining the temperature below 30° C. The biphasic solution was separated and the organic phase washed with H₂O (2×150 mL) then the solvent exchanged to EtOH under reduced pressure to give the title compound as a crude solution in EtOH (128 g, 26% w/w, 96% yield) that was used directly in the next step. ¹H NMR for purified compound (500 MHz, CDCl₃) δ 0.98 (3H, s), 1.16 (3H, s), 1.21-1.29 (2H, m), 1.51-1.66 (5H, m), 1.66-1.76 (3H, m), 1.76-1.82 (1H, m), 1.88-2.02 (2H, m), 2.26 (3H, dd), 2.40 (1H, dd), 2.80 (1H, s), 3.00 (1H, s), 3.05 (1H, dd), 3.21 (1H, dd), 3.93 (3H, s), 4.06-4.2 (1H, m), 4.39 (1H, td), 5.60 (2H, s), 5.64 (1H, t), 6.19-6.38 (2H, m), 6.70 (1H, d), 7.46 (1H, dd), 7.49-7.62 (3H, m), 7.75-7.93 (3H, m), 8.00 (1H, d), 8.66 (1H, d). MS (ESI): m/z [M+H]⁺ 683.3.

### Compound (I): (1S,4s)-4-(2-Fluoro-4-methoxy-5-(((1S,2R,3S,4R)-3-(((1-methylcyclobutyl)methyl)carbamoyl)bicyclo[2.2.1]heptan-2-yl)carbamoyl)phenoxy)-1-methylcyclohexane-1-carboxylic acid

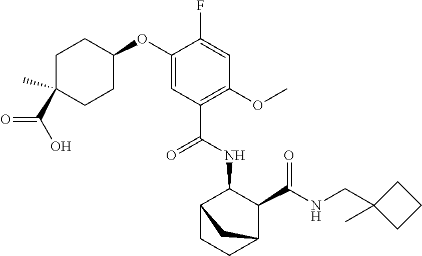 [SMILES: COc1cc(F)c(O[C@H]2CC[C@@](C)(C(=O)O)CC2)cc1C(=O)N[C@@H]1[C@H]2CC[C@H](C2)[C@@H]1C(=O)NCC1(C)CCC1]

To a solution of Intermediate 17 in EtOH (206 g, 19% w/w, 57.9 mmol) was added EtOH (385 mL) followed by 10 wt % Pd/C (3.96 g, 5% w/w). The vessel was purged with N₂ six times followed by H₂ a further six times. The vessel was pressurized to 0.4 MPa with H₂ and the reaction solution stirred for 20 h at between 2° and 30° C. The H₂ atmosphere was completely replaced with N₂ before the reaction mixture was filtered and the solids washed with EtOH (3×80 mL). A second identical batch was conducted and the collected EtOH solutions combined to give a single solution. The solvent was exchanged to EtOAc under reduced pressure maintaining the temperature below 45° C. The EtO Ac solution (280 mL) was heated to between 7° and 75° C. for 0.5 h then cooled to between 4° and 45° C. and n-heptane (475 mL) added drop-wise over 0.5 h. The mixture was stirred for 0.5 h then cooled to between 2° and 25° C. over 2 h then held for a further 2 h. The heterogenous slurry was filtered then the solids washed twice with 1:2 EtOAc/n-heptane (160 mL) prior to drying at below 45° C. for 20 h to give crude title compound as a white solid (55.7 g, 87%).

Part 1: The crude title compound (2.50 g, 4.59 mmol) was dissolved in EtOH (15.0 mL). The temperature of the solution was maintained at 25.0±2.0° C. during the drop-wise addition of water (7.50 mL) during which a precipitate formed. The heterogenous slurry was stirred for a further 1.0 h then collected via filtration. The solids were washed with a (2:3) mixture of EtOH/Water (2×5.00 mL), collected and dried under N₂ to give the title compound as a white solid (1.80 g, 72%). This material was characterized as Form A and used as seed following the method described in Part 2.

Part 2: The crude title compound (50.0 g, 91.8 mmol) was dissolved in EtOH (350 mL) then passed through a filter. EtOH (100 mL) was added to vessel then passed through the filter to give a combined EtOH solution. The temperature of the solution was maintained at 25.0±2.0° C. during the slow addition of H₂O (150 mL) over 0.5 h. The solution was stirred for a further 0.5 h then seed material from Part 1 (0.005 g, 0.1% w/w) was added. The solution was held for 6 h then cooled to 20.0±0.5° C. over 2 h, then held for a further 6 h. H₂O (150 mL) was added slowly over 6 h then the mixture held for a further 2 h prior to filtration. EtOH (45 mL) and H₂O (30 mL) was added to vessel then used to wash the filter cake. The solids were collected and dried under N₂ at below 45° C. for 12 h to give the title compound Form A as a white solid (42.2 g, 85%); ¹H NMR (500 MHz, CDCl₃) δ 0.97 (3H, s), 1.12-1.42 (5H, m), overlapping 1.25 (3H, S), 1.43-1.82 (10H, m), 1.92-2.1 (3H, m), 2.25 (3H, dd), 2.51 (2H, dd), 2.96 (1H, dd), 3.18 (1H, dd), 3.92 (3H, s), 4.12-4.28 (1H, m), 4.41 (1H, t), 5.81 (1H, t), 6.70 (1H, d), 7.86 (1H, d), 8.60 (1H, d). HRMS (ESI) m/z [M+H]⁺ calcd for C30H42FN2O6: 545.3022 found: 545.3019. The XRPD pattern (XRPD Method A) is shown in FIG. 1.

### Example 1

### Amorphous (1S,4s)-4-(2-Fluoro-4-methoxy-5-(((1S,2R,3S,4R)-3-(((1-methylcyclobutyl)methyl)carbamoyl)bicyclo[2.2.1]heptan-2-yl)carbamoyl)phenoxy)-1-methylcyclohexane-1-carboxylic acid

A solution of (1S,4s)-4-(2-Fluoro-4-methoxy-5-(((1S,2R,3S,4R)-3-(((1-methylcyclobutyl)methyl)carbamoyl)bicyclo[2.2.1]heptan-2-yl)carbamoyl)phenoxy)-1-methylcyclohexane-1-carboxylic acid (3915 mg, 7.19 mmol) in 1,4-dioxane/water 80:20 (67 mL) was frozen in liquid nitrogen and dried in a freeze dryer (Christ Alpha 2-4 LD) overnight to yield amorphous (1S,4s)-4-(2-Fluoro-4-methoxy-5-(((1S,2R,3S,4R)-3-(((1-methylcyclobutyl)methyl)carbamoyl)bicyclo[2.2.1]heptan-2-yl)carbamoyl)phenoxy)-1-methylcyclohexane-1-carboxylic acid (3915 mg, 7.19 mmol) as a white solid. The X-ray powder diffraction pattern (XRPD Method A) of this amorphous form is shown in FIG. 2.

### Example 2

### A solid dispersion of amorphous (1S,4s)-4-(2-Fluoro-4-methoxy-5-(((1S,2R,3S,4R)-3-(((1-methylcyclobutyl)methyl)carbamoyl)bicyclo[2.2.1]heptan-2-yl)carbamoyl)phenoxy)-1-methylcyclohexane-1-carboxylic acid

Polymers compatible with Compound (I) were screened to select an appropriate polymer for the solid dispersion. The following polymers were found to be stable over 4 weeks at 50° C. and 40° C./75% relative humidity (RH) with no signs of crystallisation, and were found to be suitable at 10%, 20% and 40% drug load;
Kollidon® 30 (poly(1-vinyl-2-pyrrolidone) available from BASF Pharma)
Kollidon® VA 64 (a copolymer of 1-vinyl-2-pyrrolidone and vinyl acetate in a ratio of 6:4 by mass available from BASF Pharma)
Soluplus® (polyvinyl caprolactam-polyvinyl acetate-polyethylene glycol graft co-polymer available from BASF Pharma)
Eudragit® L100-55S (methacrylic acid-ethyl acrylate copolymer (1:1) available from Evonik Industries AG)
Polyacrylic acid
AQOAT® HPMC AS-LF (hypromellose acetate succinate available from Shin-Etsu Chemical Co., Ltd.)
AQOAT® HPMC AS-HF (hypromellose acetate succinate available from Shin-Etsu Chemical Co., Ltd.)
Cellulose acetate phthalate
Polyvinyl Acetate Phthalate

Three amorphous solid dispersions of Compound (I) were prepared at drug loadings of 20%, 30% and 40% w/w according to the following process:
1. Compound (I) was weighed into a glass container and Kollidon® VA 64 (a copolymer of 1-vinyl-2-pyrrolidone and vinyl acetate in a ratio of 6:4 by mass available from BASF Pharma) was then added. The powder was then mixed for 5 minutes at 32 rpm using a Turbula® blender.
2. A hot melt extruder was first cleaned with melting beads before extruding the mixtures. The 20% drug load solid dispersion was extruded at 200° C. and filaments were collected in clean glass container. The 30% drug load solid dispersion was also extruded at 200° C., and the 40% drug load solid dispersion was extruded at 210° C. 3. The collected filaments were then milled manually using a lab mill.

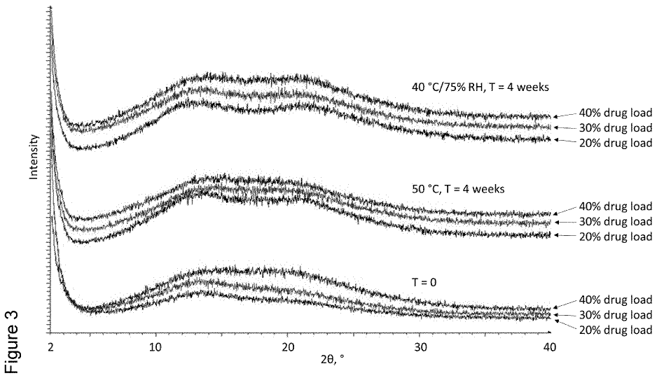

The samples were placed on stability at 40° C./75% relative humidity (RH) (Open container) and 50° C. (Closed container, ambient RH) for 4 weeks with the XRPD of the samples recorded every week. FIG. 3 shows the XRPD diffraction patterns (XRPD Method B) of each of the samples after formation, and after storage for 4 weeks at 40° C./75% relative humidity (RH) (Open container), and 4 weeks at 50° C. (Closed container, ambient RH). The XRPD patterns indicated that no crystallization of Compound (I) occurred upon storing the amorphous solid dispersions for 4 weeks at 40° C./75% relative humidity (RH) (Open container), and 4 weeks at 50° C. (Closed container, ambient RH). Dissolution experiments with the 30% drug load solid dispersion indicated improved dissolution in simulated gastric fluid pH 1.8 and Fasted State Simulated Intestinal Fluid (FaSSIF) pH 6.5 vs a granulate based on crystalline Form A material of Compound (I).

### Example 3

### Form G of (1S,4s)-4-(2-Fluoro-4-methoxy-5-(((1S,2R,3S,4R)-3-(((1-methylcyclobutyl)methyl)carbamoyl)bicyclo[2.2.1]heptan-2-yl)carbamoyl)phenoxy)-1-methylcyclohexane-1-carboxylic acid

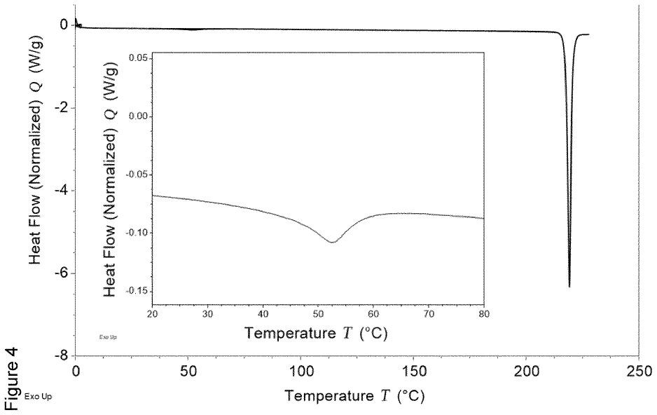

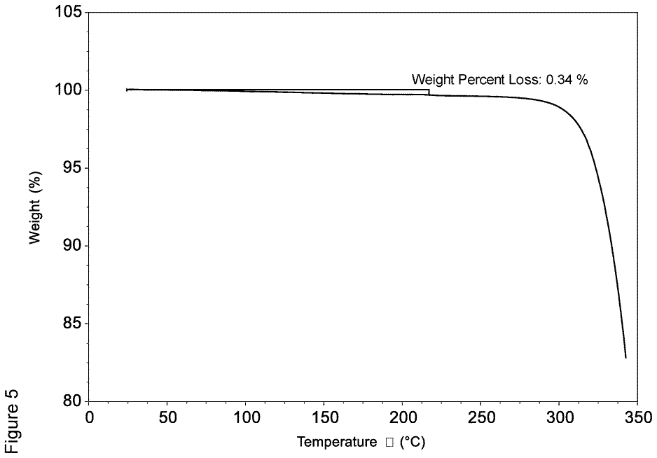

Upon heating Form A of (1S,4s)-4-(2-Fluoro-4-methoxy-5-(((1S,2R,3S,4R)-3-(((1-methylcyclobutyl)methyl)carbamoyl)bicyclo[2.2.1]heptan-2-yl)carbamoyl)phenoxy)-1-methylcyclohexane-1-carboxylic acid, DSC shows an endothermic transformation with an onset observed around 43.9° C. This endothermic peak is assigned to a solid-solid phase transition and the resulting phase at temperatures above the completion of the endothermic peak observed at around 43.9° C. is referred to as Form G. This, new, anhydrous phase Form G exists until the last endothermic peak in the DSC which corresponds to melting onset at 217.5° C. The DSC output for Form A/Form G is shown in FIG. 4. TGA analysis of Form A/Form G is shown in FIG. 5.

### Biological and Physicochemical Data for Compound (I)

RXFP1 Hu cAMP (Test A)

To screen for modulators of hRXFP1, an assay identifying compounds that stimulate CAMP production via the Gs-coupled hRXFP1 receptor was used. cAMP HiRange HTRF kit (available from CisBio Bioassays, France; catalogue number 62AM6PEJ) was employed in large according to manufacturer's recommendations for detection of cAMP. The HTRF method is a competitive immunoassay between native cAMP produced by cells and cAMP labeled with the dye d2. The tracer binding is visualized with a cryptate labeled antibody for cAMP and the signal is thus inversely proportional to the amount of produced cAMP.

### Preparation of Assay Reagents:

Assay buffer: HBSS (ThermoFisher, 14065) with 5 mM Hepes (ThermoFisher, 15630) pH 7.4 containing 0.1% BSA (Sigma, A8806)

Cells: Jump-In™ T-REX™ CHO-K1 Cells (ThermoFisher) stably transfected with human RXFP1 was employed. Cells were induced to express human RXFP1 by treatment with 10 ng/ml doxycycline for 24 h. Cells were then cryopreserved for long term storage. At the start of each experiment, cells were thawn, washed with PBS and resuspended in assay buffer to 1.875*10\hat{ }5 cells/ml

CAMP standard: stock standard cAMP provided in the CisBio kit was diluted in assay buffer to a top final concentration of 2.8 μM in the assay.

HTRF detection reagents: cAMP-d2 and anti-cAMP cryptate reconstituted according to CisBio instructions were diluted 1:40 in lysis buffer provided with the HTRF-kit.

### Step by Step Procedure for Running the Assay:

1. 40 nL test compounds dissolved in DMSO were aquostically dispensed (Labcyte Echo) to white 384-well plates (Greiner; 784075), sealed and stored at room temperature until assayed.

2. 40 nL 200 nM Relaxin-2 in DMSO (1 nM final concentration) was added to 100% control wells and 40 nL DMSO added to 0% wells with Echo acoustic dispenser at the day of assay.

3. 4 μL assay buffer with 1 mM IBMX (0.5 mM final concentration) to block phosphodiesterases was added with Multidrop Combi (ThermoFisher).

4. 4 μL cell solution at 1.875*10\hat{ }5 cells/ml was added with Multidrop Combi to give 750 cells/well.

5. 45 min incubation at room temperature.

6. 4 μL cAMP-d2 in lysis buffer was added with Multidrop Combi.

7. 4 μL anti-cAMP cryptate in lysis buffer was added with Multidrop Combi

8. 2 h incubation at room temperature

9 Homogenous Time-Resolved Fluorescence (HTRF) signal was detected with an Envision (PerkinElmer) or Pherastar (BMG Labtech) reader (λex=340 nm, λem=665 and 615 nm).

Using a cAMP standard curve, HTRF data was converted to amount cAMP produced in the samples which was subsequentially used for calculation of concentration responses. Concentration response data were fitted with a four parameter logistic fit, the Hill equation. The results from the assay are reported in Table 1 as EC₅₀ (μM) and S_{inf} (%).

EC₅₀ is defined as the concentration at which the stimulatory activity reaches 50% of its maximum level. Where the assay was run multiple times for the same compound, the geometric mean is reported.

S_{inf} is the fitted activity level, efficacy, at infinite concentration of test compound. To facilitate comparison of efficacy data, efficacy was normalized to % effect of the response stimulated by a saturating concentration of relaxin (1 nM). Where the assay was run multiple times for the same compound, the arithmetic mean is reported.

### Human Plasma Protein Binding (Test B)

The assay was conducted according to the Human Plasma Protein Binding Assay described in pages 167-170 of Wernevik, J. et al., “A Fully Integrated Assay Panel for Early Drug Metabolism and Pharmacokinetics Profiling”, Assay and Drug Development Technologies, 2020, 18(4), 157-179. Data are reported in Table 1 as fraction unbound (fu) (% free). Where the assay was run multiple times for the same compound, the arithmetic mean is reported.

### Human Liver Microsomal Stability (Test C)

The assay was conducted according to the Human Liver Microsome Stability Assay described in pages 170-174 of Wernevik, J. et al., “A Fully Integrated Assay Panel for Early Drug Metabolism and Pharmacokinetics Profiling”, Assay and Drug Development Technologies, 2020, 18(4), 157-179. Data are reported in Table 1 as CLᵢₙₜ (μl/min/mg protein). Where the assay was run multiple times for the same compound, the arithmetic mean is reported.

### Human Hepatocyte Stability (Test D)

The metabolic stability of compounds in human hepatocytes was assessed using the following protocol:

1. Prepare 10 mM stock solutions of compound and control compounds in appropriate solvent (DMSO). Place incubation medium (L-15 Medium) in a 37° C. water bath, and allow warming for at least 15 minutes prior to use.

2. Add 80 μL of acetonitrile to each well of the 96-well deep well plate (“Quenching plate”).

3. In a new 96-well plate, dilute the 10 mM test compounds and the control compounds to 100 μM by combining 198 μL of acetonitrile and 2 μL of 10 mM stock solution.

4. Remove a vial of cryopreserved (less than −150° C.) human hepatocytes (Liverpool™ 10-Donor Human hepatocytes obtained from Bioreclamation IVT (Product No. S01205)) from storage, ensuring that vials remain at cryogenic temperatures until thawing process ensues. As quickly as possible, thaw the cells by placing the vial in a 37° C. water bath and gently shaking the vials. Vials should remain in water bath until all ice crystals have dissolved and are no longer visible. After thawing is complete, spray vial with 70% ethanol, transfer the vial to a bio-safety cabinet.

5. Open the vial and pour the contents into the 50 ml conical tube containing thawing medium. Place the 50 mL conical tube into a centrifuge and spin at 100 g for 10 minutes (room temperature). Upon completion of spin, aspirate thawing medium and resuspend hepatocytes in enough incubation medium to yield ~1.5×10⁶ cells/mL.

6. Using Cellometer® Vision, count cells and determine the viable cell density. Cells with poor viability (<80% viability) are not acceptable for use. Dilute cells with incubation medium to a working cell density of 1.0×10⁶ viable cells/mL.

7. Transfer 247.5 μL of hepatocytes into each well of a 96-well cell incubation plate. Place the plate on Eppendorf Thermomixer Comfort plate shaker to allow the hepatocytes to warm for 10 minutes.

8. Add 2.5 μL of 100 μM test compound or control compounds into an incubation well containing cells to initiate the reaction.

9. Incubate the plate at 37° C. and 900 rpm on an Eppendorf Thermomixer Comfort plate shaker. At 0.5, 5, 15, 30, 45, 60, 80, 100 and 120 min, transfer 20 μL of the incubated mixture to a separate “Quenching plate”, then mix the sample by vortex for 2 min.

10. Centrifuge the quenching plates for 20 minutes at 4,000 rpm. Transfer 30 μL of supernatant of each compound into a 96-well analysis plate. 4 compounds are pooled together into one cassette. Then dilute the pooled sample by adding of 180 μl of pure water. All incubations are performed in singlicate.

All calculations were carried out using Microsoft Excel. Peak areas were determined from extracted ion chromatograms. In vitro intrinsic clearance (in vitro Clᵢₙₜ, in μL/min/10⁶ cells) of parent compound was determined by regression analysis of the Ln percent parent disappearance vs. time curve. The in vitro intrinsic clearance (in vitro Clᵢₙₜ, in μL/min/10⁶ cells) is reported in Table 1, and was determined from the slope value using the following equation:

$$in vitro {Cl}_{int} = kV / N$$

$$V = incubation volume ( 0.25 mL ) ;$$

$$N = number of hepatocytes per well ( 0.25 \times {10}^{6} cells )$$

Where the assay was run multiple times for the same compound, the geometric mean is reported.

### Rat Hepatocyte Stability (Test E)

The assay was conducted according to the Rat Hepatocyte Stability Assay described in pages 170-174 of Wernevik, J. et al., “A Fully Integrated Assay Panel for Early Drug Metabolism and Pharmacokinetics Profiling”, Assay and Drug Development Technologies, 2020, 18(4), 157-179. Data are reported in Table 1 as mean Clᵢₙₜ (μl/min/10⁶ cells). Where the assay was run multiple times for the same compound, the geometric mean is reported.

### Solubility (pH 7.4) (Test F)

The assay was conducted according to the Solubility Assay described in pages 164-167 of Wernevik, J. et al., “A Fully Integrated Assay Panel for Early Drug Metabolism and Pharmacokinetics Profiling”, Assay and Drug Development Technologies, 2020, 18(4), 157-179. Data are reported in Table 1 as solubility (μM). Where the assay was run multiple times for the same compound, the arithmetic mean is reported.

TABLE 1
Assay data for Compound (I)

| | | Test B | Test C | | | |
|---|---|---|---|---|---|---|
| | | Human | Human Liver | Test D | Test E | |
| | | Plasma | Microsomal | Human | Rat | Test F |
| | | Protein | Stability | Hepatocyte | Hepatocyte | Solubility |
| Test A | | Binding | Clᵢₙₜ | Stability Clᵢₙₜ | Stability Clᵢₙₜ | pH 7.4 |
| EC₅₀ (μM) | S_{inf} (%) | (% free) | (μl/min/mg) | (μL/min/10⁶ cells) | (μL/min/10⁶ cells) | (μM) |
| 0.017 | 109 | 4.3 | 23 | 4.8 | 11 | >500 |

Human RXFP1 cGMP Production Assay (Test G)

To profile compounds for RXFP1 agonist activity with respect to cGMP production, the Green GENIe cGMP Assay (Montana Molecular; catalogue number D800G) was employed. The assay is based on an mNeonGreen fusion protein fluorescent biosensor delivered to mammalian cells in a BacMam vector. Fluorescence is reduced when cGMP is bound to the biosensor.

### Preparation of Assay Reagents:

Assay buffer: DPBS (Gibco; 14040133) containing 0.1% BSA (Sigma; A8806)

Cells: HEK293s cells stably transfected with human RXFP1 in pIRESneo3 was employed. Cells were cultured in DMEM medium (Gibco; 31966) with 10% FBS complemented with 0.8 mg/mL to maintain RXFP1 expression.

### Step by Step Protocol for Running the Assay:

### Day 1

1. Cells were splitted one day ahead of transduction and seeded to 63 000 cells/cm² in DMEM medium with 10% FBS without antibiotics in a tissue culture flask.

### Day 2

2. After PBS wash cells were detached using accutase (Gibco; 1737341), resuspended in medium and collected in a 50 mL tube.
3. Cells were counted with a CEDEX (Innovatis) and diluted with medium to 267 000 cell/mL.
4. A viral transduction mastermix was prepared by mixing reagents in the following proportions for a single well:
   6 μL GENIe BacMAM vector
      0.2 μL 500 mM sodium butyrate
         13.8 μL DMEM Medium with 10% FBS
20 μL total volume
5. Cells and transduction mastermix were mixed in proportions 30 μL cells and 20 μL mastermix for a single well.
6. 50 μL cell-transduction mix from above was dispensed per well into Black poly-D-lysine coated μclear 384-well plates (Greiner; 781946).
7. Plate was incubated in the dark at 37° C., 5% CO₂ for 24 h.

### Day 3

8. Medium was removed from plates using a Bluewasher (BluCatBio).
9. 20 μL assay buffer was added with a Multidrop Combi (ThermoFisher).
10. Plate was incubated in the dark at room temperature for 30 min prior to assaying.
11. Plates were assayed using a FLIPR Tetra (Molecular Devices): 10 μL compound diluted with assay buffer was added to each well by the FLIPR Tetra and measuring green fluorescence over time for up to 3 h.

Data was processed using Screener software (Genedata AG). After subtraction of background fluorescence (before addition of compounds), area under curve values from 0 to 90 min after compound addition was used for calculation of responses. Concentration response data were fitted with a four parameter logistic fit and EC₅₀ values (nM) are reported in Table 2.

### Human RXFP1 Phospho-ERK Assay (Test H)

To profile compounds for RXFP1 agonist activity with respect to ERK phosphorylation, the advanced phospho-ERK (Thr202/Tyr204) cellular kit (CisBio; 64AERPEH) was employed. The assay uses two antibodies. One labeled with a donor fluorophore (Eu cryptate), a second with an acceptor (d2). The first antibody specific binds to phosphorylated ERK, the second binds another motif of ERK and independently of its phosphorylation state. ERK phosphorylation enables immune-complex formation involving the two antibodies, thereby generating a FRET signal. Its intensity is proportional to the concentration of phosphorylated ERK in the sample. Assay was performed according to manufacturers recommendations.

### Preparation of Assay Reagents:

Cells: HEK293s cells stably transfected with human RXFP1 in pIRESneo3 was employed. Cells were cultured in DMEM medium (Gibco; 31966) with 10% FBS complemented with 0.8 mg/mL to maintain RXFP1 expression. Assay was performed on cells kept in continuous culture.

Dilution of test compounds: Compounds were diluted to desired concentrations with serum-free DMEM without phenol red (Gibco; 31053-038). DMSO concentration was adjusted to 0.4%

Antibody mix: The Eu and d2 labelled anti ERK1/2 antibodies were separately diluted 20-fold with detection buffer provided in the kit. Shortly prior to the experiment, equal volumes of each diluted antibody solution were combined to an antibody mix.

### Step by Step Protocol for Running the Assay:

### Day 1

1. Cells were detached from culture flasks using accutase (Gibco; 1737341), resuspended in DMEM medium without phenol red containing 10% FBS and collected in a 50 mL tube.
2. Cells were counted with a CEDEX (Innovatis) and diluted with the medium above to 320 000 cell/mL.
3. 100 μL cell suspension was dispensed per well into Black μclear poly-D-lysine coated μclear 96-well plates (Greiner; 655946).
4. Plates were incubated at 37° C., 5% CO₂ for 24 h.

### Day 2

5. Serum starvation: Medium was removed and replaced with 50 μL serum-free DMEM without phenol red. Plates were incubated at 37° C., 5% CO₂ for 5 h.
6. 50 μL test compound solutions were added per well.
7. Plates were incubated at room temperature for 5 min.
8. Stimulation was stopped by rapidly removing medium and adding 50 μL lysis buffer (diluted to 1× final concentration prior to addition) per well.
9. Plates were transferred to −80° C. and lysates frozen.

### Day 3

10. Plates were thawed and shaken at room temperature for 30 min.
11. Cells lysates were homogenized by pipetting.
12. 16 μL homogenate per well was transferred to white low volume 384-well plates (Greiner; 784075)
13. 4 μL antibody mix was added per well.
14. Plates were incubated at room temperature in the dark for 4 h.
15. Homogenous Time-Resolved Fluorescence (HTRF) signal was detected with a Pherastar (BMG Labtech) reader (λex=340 nm, λem=665 and 615 nm).

HTRF ratio data was processed using Screener software (Genedata AG). Concentration response data were fitted with a four parameter logistic fit and EC₅₀ value (nM) reported in Table 2.

TABLE 2
Assay Data

| | Test G | Test H |
|---|---|---|
| | cGMP assay | phospo-ERK assay |
| Compound | EC₅₀ (nM) | EC₅₀ (nM) |
| Compound (I) | 50 | 6.3 |
| relaxin2 | 0.085 | 0.038 |

### Solubility of Amorphous Compound (I) in pH 1.2 SGF (Test I)

### Preparation of Amorphous Nanosuspension of Compound (I)

A 100 mM stock solution of Compound (I) was prepared in DMA and miglyol 812 (9.44 mg in 140.2 μL of DMA and 23.6 μL in miglyol 812) by ultrasonic treatment and stirring. 10 μL of the stock solution was rapidly injected into 990 μL of stabilizer solution containing 0.2% w/w PVPK30 and 0.2% w/w Pluronic F127 and 0.25 mM SDS aqueous solution under a few seconds of ultrasonic treatment to make a 1 mM amorphous nanosuspension. This formulation is further diluted to 0.25 mM with purified water.

### Solubility Experiment

The bulk concentration in equilibrium with amorphous particles is determined at 37° C. by a turbidimetric method (described in Lindfors et. al., Langmuir, 2006, 22(3), 911-916). Small volumes of drug suspension are successively added directly to a fluorescence cuvette containing 2000 μL simulated gastric fluid pH 1.2 (SGF) and are then mixed to give the desired concentrations. The light scattering intensity at 700 nm is recorded at a scattering angle of 90° as a function of total drug concentration. As a light scattering setup, a Perkin-Elmer LS 55 Luminescence Spectrometer is used, setting both the emission and excitation wavelengths to 700 nm. Slit excitation is set to 2.5 nm and slit emission is set to 5 nm. Particles are allowed to dissolve for at least 30 seconds prior to measurements starting. Data are then collected for 1 minute and a mean light scattering intensity is calculated. The solubility examination was performed within 45 min of the formation of the amorphous nanosuspension.

| Volume of 0.25 | | |
|---|---|---|
| mM suspension | Total concentration of | Mean light scattering |
| added | Compound (I) (uM) | intensity (a.u.) |
| 0 | 0.0 | 0.17 |
| 10 | 1.2 | 0.16 |
| 10 | 2.5 | 0.28 |
| 10 | 3.7 | 0.33 |
| 20 | 6.1 | 0.33 |
| 20 | 8.5 | 1.71 |
| 20 | 10.8 | 4.94 |
| 20 | 13.0 | 8.19 |
| 20 | 15.3 | 11.77 |
| 20 | 17.4 | 15.33 |
| 20 | 19.6 | 18.47 |

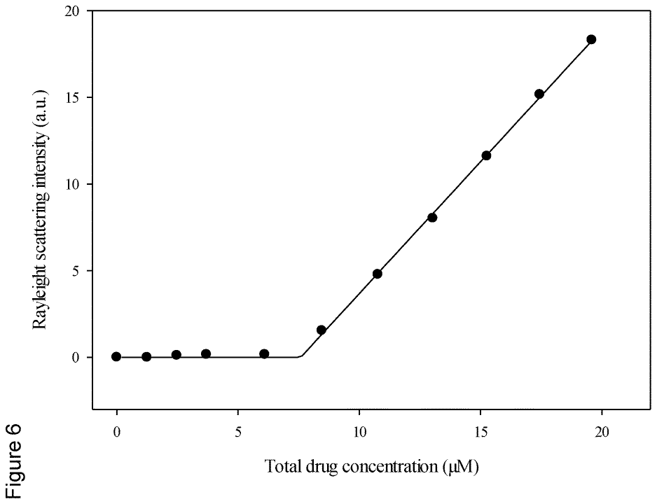

Light scattering was plotted as a function of total concentration of Compound (I). At low concentrations the particles dissolve, while at concentrations above the solubility, the particles remain suspended, leading to an almost linear increase in light scattering intensity as a function of total concentration. Extrapolation of a linear line fitted to the high concentration data to zero intensity was used to estimated the amorphous solubility of Compound (I) in pH 1.2 SGF at 37° C., and this was determined to be 8 μM. The light scattering intensity as a function of total concentration is shown in FIG. 6.

### Solubility of Crystalline Form a of Compound (I) in pH 1.2 SGF (Test J)

Crystalline solubility in simulated gastric fluid pH 1.2 (SGF) was measured using a miniaturized shake-flask method (24 h agitation at 37° C.). An excess of Form A of Compound (I) was weighed into vials, 1.2 mL of pH 1.2 SGF was added, and the samples were ultrasonicated for 45 min.

The samples were agitated at 37° C. for 24 hours in an Eppendorf shaker, agitation speed 1000 rpm. The samples were then centrifuged at 10000 RPM for 15 minutes, 37° C. Approximately 500 μL of the supernatant was withdrawn with automatic pipette to a new vial and centrifuged again for 15 min at 10000 rpm, 37° C. Then from the second centrifugation, samples were taken with automatic pipette to final dilution. pH was measured in the equilibrated remaining sample solution after the second centrifugation. XRPD was used to confirm that no form change had occurred. UHPLC was used to quantify concentrations in the samples, and the solubility of Form A of Compound (I) in pH 1.2 SGF at 37° C. was found to be 0.4 μM.

Those skilled in the art will appreciate that the assays described above may be performed using alternative equipment and minor variations to the protocol without significantly affecting the results.

This description and its specific examples, while indicating certain embodiments, are intended for purposes of illustration only. This disclosure, therefore, is not limited to the illustrative embodiments described in this specification, and may be variously modified. In addition, it is to be appreciated that various embodiments that are, for clarity reasons, described in the context of separate embodiments, also may be combined to form a single embodiment. Conversely, various embodiments that are, for brevity reasons, described in the context of a single embodiment, also may be combined to form sub-combinations thereof.

Any publications disclosed within the specification are hereby incorporated by reference.

1. Amorphous Compound (I)

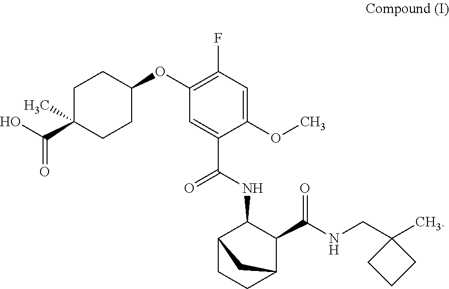 [SMILES: COc1cc(F)c(O[C@H]2CC[C@@](C)(C(=O)O)CC2)cc1C(=O)N[C@@H]1[C@H]2CC[C@H](C2)[C@@H]1C(=O)NCC1(C)CCC1]

2. A pharmaceutical composition comprising amorphous Compound (I) and a pharmaceutically acceptable excipient.

3. The pharmaceutical composition of claim 2, wherein at least 80% by weight of Compound (I) in the pharmaceutical composition is amorphous.

4. A solid dispersion comprising amorphous Compound (I).

5. The solid dispersion of claim 4, further comprising one or more polymers.

6. The solid dispersion of claim 4 or 5, wherein at least 80% by weight of Compound (I) in the solid dispersion is amorphous.

7. The solid dispersion of any one of claims 4 to 6, wherein less than 20% by weight of Compound (I) in the solid dispersion is crystalline.

8. The solid dispersion of any one of claims 4 to 7, wherein Compound (I) is present in an amount of from 5% by weight to 95% by weight.

9. The solid dispersion of any one of claims 4 to 8, wherein the one or more polymers are present in an amount of from 5% by weight to 95% by weight.

10. The solid dispersion of any one of claims 4 to 9, wherein the one or more polymers are water-soluble.

11. The solid dispersion of any one of claims 4 to 10, wherein the one or more polymers are selected from poly(1-vinyl-2-pyrrolidone), a copolymer of 1-vinyl-2-pyrrolidone and vinyl acetate, polyvinyl caprolactam-polyvinyl acetate-polyethylene glycol graft co-polymer, methacrylic acid-ethyl acrylate copolymer, polyacrylic acid, hypromellose acetate succinate, cellulose acetate phthalate, and polyvinyl acetate phthalate.

12. The solid dispersion of any one of claims 4 to 10, wherein the one or more polymers are a copolymer of 1-vinyl-2-pyrrolidone and vinyl acetate, optionally a copolymer of 1-vinyl-2-pyrrolidone and vinyl acetate in a ratio of 6:4 by weight.

13. A pharmaceutical composition comprising the solid dispersion of any one of claims 4 to 12.

14. The pharmaceutical composition of any one of claims 2, 3 and 13, wherein the pharmaceutical composition is in the form of a tablet or capsule.

15. The pharmaceutical composition of any one of claims 2, 3, 13 and 14, wherein the pharmaceutical composition is suitable for oral administration.

16. The compound, solid dispersion or pharmaceutical composition of any one of the preceding claims for use in therapy.

17. The compound, solid dispersion or pharmaceutical composition of any one of claims 1 to 15 for use in the treatment of a subject with a disease or condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension.

18. A crystalline form of Compound (I), wherein the crystalline form has a melting onset at about 217.5° C.

19. A crystalline form of Compound (I), obtainable by heating crystalline Form A of Compound (I) to a temperature above about 43.9° C., optionally above about 45° C. optionally above about 50° C., optionally above about 55° C., optionally above about 60° C., optionally above about 65° C.

20. A method of preparing the crystalline form of claim 18 or 19, comprising heating crystalline Form A of Compound (I) to a temperature above about 43.9° C., optionally above about 45° C. optionally above about 50° C., optionally above about 55° C., optionally above about 60° C., optionally above about 65° C.

21. The crystalline form of claim 19, or the method of claim 20, wherein Form A of Compound (I) has an X-ray powder diffraction pattern (Cu K_{α} radiation) with at least five specific peaks at about 2-theta=7.5, 10.7, 12.8, 14.5 and 15.8°.

22. The crystalline form of claim 19, or the method of claim 20, wherein Form A of Compound (I) has an X-ray powder diffraction pattern (Cu K_{α} radiation) substantially the same as the X-ray powder diffraction pattern shown in FIG. 1.

23. A pharmaceutical composition comprising the crystalline form of any one of claims 18 to 22 and a pharmaceutically acceptable excipient.

24. The crystalline form of any one of claims 18 to 22 for use in the manufacture of a medicament for the treatment of a disease or condition selected from heart failure, heart failure with preserved ejection fraction, heart failure with mid-range ejection fraction, heart failure with reduced ejection fraction, heart failure with pulmonary hypertension, chronic kidney disease, acute kidney injury, hypertension, and resistant hypertension in a human patient.
